(19) United States
(12) Patent Application Publication
(10) Pub. No.: US 2026/0266811 A1
(43) Pub. Date: Sep. 10, 2026

(54) A METHOD FOR DETECTING AN ANALYTE

(71) Applicant: Psyros Diagnostics Limited, Sandwich Kent (GB)

(72) Inventors: Steven Andrew Ross, Aldington Kent (GB); Mark Thomas Gatton Swayne, Herne Bay, Kent (GB); Aileen Jane McGettrick, Herne Bay Kent (GB); Paul Brendan Monaghan, Sandwich Kent (GB); Julie Richards, Faversham Kent (GB)

(21) Appl. No.: 19/166,443

(22) PCT Filed: Mar. 15, 2024
(86) PCT No.: PCT/GB2024/050716

§ 371 Date: Sep. 17, 2025

(30) Foreign Application Priority Data
Mar. 17, 2023 (GB) 2303923.3

(51) Int. Cl. G01N 33/543; G01N 21/64

(52) U.S. Cl. CPC G01N 33/54313; G01N 21/6428; G01N 2021/6439

(57) ABSTRACT

The present invention relates to a method for detecting an analyte in a sample suitable for detecting the analyte over a range of concentrations, the method comprising steps (i)-(v). Thus, the present invention provides a method for detecting an analyte in a sample in which only the reporter reagents in the vicinity of the surface of the substrate results in a signal, the signal being a local region of the optical component in the second optical state. It is the set of local regions of the optical component in the second optical state that are detected.

The present invention relates to a method for detecting an analyte, and particularly to a method for increasing the concentration range at which the analyte can be detected.

There are many techniques available for the measurement of biologically relevant parameters in human samples, such as blood, plasma, serum, tissue etc. A common method is to use a capture reagent that binds to the target of interest, and a reporter reagent that has a label of some sort. The capture reagent can be bound to a solid phase, such as a microtitre plate, a bead or a membrane. When the sample is incubated with the capture reagent, the analyte binds to the capture reagent. The reporter reagent also binds to the analyte. Excess reporter can then be removed (by washing) and the quantity of reporter reagent can be measured, thus giving a measure of the amount of analyte present in the sample. There are many different variations on how these types of binding assay can be carried out. For example, the analyte can bind to the capture reagent first, then the reporter can be added in a separate step, or the analyte can bind to the reporter first, and then bind to the capture reagent.

There are a wide range of reagents that can be used as capture and reporter in binding assays of this type, including nucleic acids, carbohydrates, antigens, peptides, proteins and antibodies. There are also a wide range of target analytes, including peptides, proteins, antibodies, nucleic acids, cells, carbohydrates, small molecules, therapeutic drugs, drugs of abuse, steroids, hormones, lipids etc.

Assays that use antibodies are commonly termed immunoassays. Immunoassays can take a number of formats. For example, when a capture antibody is used to capture the analyte and a reporter antibody is used to generate a measurable signal, this is commonly called a sandwich immunoassay. Alternative formats are known, where the binding agent is attached to a solid phase and target analyte competes in solution with a labelled reagent that also binds to the binding agent. In the absence of analyte, a high signal is obtained, since high levels of the labelled reagent bind. In the presence of analyte, a fraction of the binding sites are blocked, thus less of the labelled reagent binds and the signal is reduced. These assays are commonly known as inhibition or competition assays. Several types of competition assay are known. For example, an antibody could be bound to the solid phase and labelled analyte (or analogue of the analyte) can compete for the binding sites on the antibody. Alternatively, an analogue of the analyte could be immobilised, and a labelled antibody could bind to this surface. In the presence of analyte in the sample, this will bind to the antibody in solution, preventing binding to the surface, and signal will be diminished.

There are many formats for assays, and many different types of label that can be employed. For example, assays can be heterogeneous, in that excess label is removed before the measurement takes place, for example by the use of wash steps. Removal of excess label can also be achieved by flowing sample and reporter past a capture area. This approach is used in immunochromatographic or lateral flow strips, which are used in rapid testing for infectious disease tests and pregnancy tests, for example. Alternatively, homogeneous assays are known, where the excess reporter is not removed. Homogeneous assays tend to rely on the close proximity of capture and reporter creating some form of signal. One example of a homogeneous assay is an agglutination assay, where particles bind together in solution. The agglutinated particles cause scattering of light, which can be measured by turbidimetry or nephelometry. A further example of a homogeneous assay using particles is the luminescent oxygen channeling immunoassay (LOCI), described in further detail below.

Another example of a homogeneous assay is the fluorescence resonance energy transfer (FRET), where the capture and reporter reagents are donor and acceptor fluorophores respectively, and excitation of the donor leads to energy transfer to the acceptor, followed by light emission.

One homogeneous assay format that functions in whole blood, without removal of cellular material is the pyro-optical immunoassay. Capture antibody is coated on to pyroelectric polyvinylidene PVDF sensor and carbon particles are used as the reporter. Signal is generated by illuminating the sample with light, causing localised heating of the particles. Those bound to the sensor transfer energy to the pyroelectric sensor, causing a thermal stress that is detected as an electrical signal. The more carbon bound, the greater the signal.

The label that is attached to the reporter binding agent can be an agent that absorbs light, such as a dye, gold particle or dyed latex microsphere. Larger particles can, in principle, absorb more light, and generate more signal. However, there is a size limit where particulate labels become impractical to use in assays, as described in more detail below. Luminescent labels are also known, such as fluorescent, chemiluminescent, bioluminescent and electrochemiluminescent labels. Luminescent labels have also been encapsulated in particles for certain types of assay. Amplification of the signal may also be performed by using enzymatic or catalytic reactions. Enzymes can be used to convert a substrate from a leuco dye to a coloured form, or into a fluorescent or luminescent form. It is common for the excess enzyme to be removed using wash steps before the substrate is added, so that signal is generated only by the enzyme that is bound specifically to the analyte.

Immunoassays that do not use labels are also known, such as assays that use surface plasmon resonance as the signal transduction method. However, label free assays tend to lack the sensitivity of assays that use labels to enhance the signal.

Further information on the field of immunoassays can be found in “The Immunoassay Handbook: 4th Edition: Theory and Applications of Ligand Binding, ELISA and Related Techniques”, Ed. D. Wild, Elsevier Science, 2013.

All binding assays, including immunoassays, have limitations in terms of the minimum and maximum concentrations of analyte that can be reliably measured.

The signal maximum is generally limited by factors such as the total amount of capture antibody available to bind the analyte and the total amount of reporter antibody to generate signal. If the capture antibody is immobilised on a solid-phase, then the surface area of the solid-phase can limit the upper limit of detection. Additionally, some signal transduction techniques can be prone to saturation, such as colorimetric methods, depending on the pathlength that the light needs to take through the sample. Luminescent methods are less prone to saturation, as the gain on the detector can be attenuated to deal with higher levels of light emission. When all of the antibody binding sites in a heterogeneous assay are filled with analyte, then the maximum signal is reached and the system has saturated. The excess analyte will normally be removed in a wash step before the reporter is added. Homogeneous assays can also suffer from an effect known as high-dose hook, where the concentration of analyte is greater than the effective concentration of capture and/or reporter antibody. In this instance, at very high concentrations, all of the binding sites on the capture and the reporter can be blocked and the assay signal can be diminished, giving erroneous results.

The lower level of detection is governed by a number of different factors. Generally, all assays will be affected by attributes such as the quality of the antibodies being used (affinity and specificity) and cross-reactivity of the antibodies with related analytes. The lower limit of detection is also dependent upon the assay set-up and factors which affect the signal-to-noise ratio of the system design. For example, in a standard enzyme-linked immunosorbent assay (ELISA), capture antibody is coated onto the surface of a 96-well microtitre plate, and then sample is incubated in the wells, leading to the analyte being captured. The wells are washed, and then reporter is added in excess, binding to the captured analyte. Excess reporter is then washed away and substrate is added that can react with the enzyme, being converted into an active form. For example, a non-coloured leuco dye, such as 2,2′-azino-bis(3-ethylbenzothiazoline-6-sulphonic acid) (ABTS) can be converted by horseradish peroxidase into an oxidised green form in the presence of hydrogen peroxide. When the analyte is present in very low quantities (e.g. less than 1 picomolar), there will only be very small quantities of enzyme bound to the surface of the well. The ABTS reacts with the enzyme to generate the green form, then diffuses into the bulk of the fluid, generating a solution that is so dilute that it cannot be distinguished from background signal. Auto-conversion of substrate can also generate colour that interferes with the measurement. Similarly, other detection methods, such as fluorescence can suffer from interfering factors and auto-fluorescence of components in the sample or the reaction wells.

Another confounding factor in immunoassays can be non-specific binding of reporter reagents to the capture surface. For example, in the ELISA assay described above, the microtitre well is coated in a layer of protein, some of which can become de-natured during the coating process. It is not uncommon for reporter to bind to areas of the capture surface during the assay. If this reporter turns over substrate to contribute to the overall signal, it is not possible to distinguish the signal due to specifically bound reporter from that which is bound non-specifically. Non-specific binding can also be promoted by many of the components present in the original sample, which can bind to the capture surface during the initial incubation, modifying the surface properties of the capture layer, creating a surface that can bind the reporter. Minimising non-specific binding of reporter involves careful optimisation of all reagents and reaction conditions used during the assay, including antibodies, detergents, temperature and ionic strength.

Generally, the limit of detection of traditional immunoassays is around 0.1 picomolar to 1 nanomolar, depending upon the assay methodology. Developing assays with very low detection limits using traditional approaches often requires a great deal of optimisation, with stringent wash steps to reduce non-specific binding and maximise signal-to-noise. Additionally, the capture surface is often small in relation to the sample volume to ensure that the signal is high enough relative to the background.

One approach that has been used to circumvent issues relating to signal-to-noise and improve detection limits is to measure individual binding events, and to count these binding events as “on” or “off” events, if the measurement is above a localised threshold. In this way, much of the background noise can be eliminated. Analogies can be drawn with digitisation of audio or communication signals. These digital assays have been shown to reach detection limits that were previously unattainable using traditional analogue methods. For example, detection limits in the low femtomolar (10⁻¹⁵ mol/L) and even attomolar (10⁻¹⁸ mol/L) range have been reported. See for example “Evaluation of highly sensitive immunoassay technologies for quantitative measurements of sub-pg/mL levels of cytokines in human serum”, Yeung et al., Journal of Immunological Methods, 2016, 437, 53 and “Digital Detection of Biomarkers Assisted by Nanoparticles: Application to Diagnostics”, Cretich et al., Trends in Biotechnology, 2015, 33, 343.

The majority of labels/reporters used in assays (such as fluorophores, dyes etc.) are not visible individually using wide-field microscopy, even under high magnification, as their size is below the diffraction limit of visible light. Thus, the presence of these labels can only be measured as a bulk phenomenon, not by counting each label. In contrast, particulate labels, such as latex particles, can in theory be visualised by wide-field optical microscopy if they are above a certain size. Depending upon the optical set-up, it is possible to start visualising particles when they have a diameter of several hundred nanometres and above, depending on the numerical aperture and type of microscope.

However, using particles of this size as labels to monitor individual binding events on a capture surface (such as an antibody-antigen interaction) becomes impractical for a number of reasons. For example, particles of this size diffuse very slowly in comparison to other types of label, impairing the rate of the reaction at a planar surface. They also start to exhibit macroscopic buoyancy effects and will sediment or float if the density of the particle is significantly different to the medium that they are contained in, which can also cause issues with assay format. Particles of this size are particularly prone to binding non-specifically to surfaces, causing high background, which is difficult to remove. Finally, excess particles must be removed, requiring wash steps. However, large particles start to suffer from shear effects in the presence of fluid flow, where the shear force on the particle becomes greater than the rupture strength of the antibody-antigen interaction (approximately 60-250 pN), leading to particles being washed away (see “Rapid Femtomolar Bioassays in Complex Matrices Combining Microfluidics and Magnetoelectronics”, Mulvaney et al., Biosensors and Bioelectronics, 2007, 23, 191).

Examples of digital assays include the Quanterix Single Molecule Array (SIMOA) system and the Merck Millipore Single Molecule Counting (SMC) system.

The Quanterix SIMOA system uses antibody-coated paramagnetic beads to capture analyte from solution. The magnetic beads are then washed, and reporter antibody labelled with enzyme is added. The quantity of beads is sufficient that the probability of having more than one analyte and reporter per bead is minimised. The beads are washed again, then loaded into an array of microwells that can only hold one bead per well. The volume of the microwells is on the femtolitre scale. If the bead has enzyme attached, then fluorogenic substrate in the well is turned over. The small dimensions of the well prevent the fluorescent product from becoming too diffuse. Each well is then counted as an “on” or “off” event if the fluorescence is above a threshold value.

The SMC system is used in the Erenna and SMCxPRO systems from Merck Millipore. The fundamental measurement technique in both systems is the same. Magnetic beads, coated in capture antibody, are used to capture the target analyte in a sandwich assay. Fluorescently labelled reporter antibody also binds to the bead, in the presence of analyte. The beads are pulled down with a magnet and excess fluorescently-tagged reporter is washed away. An eluting buffer is then added that causes dissociation of the sandwich complex, which is then transferred to a measurement vessel. The presence of the fluorescent tag is then measured using a confocal fluorescent microscope that sequentially interrogates small volumes of the sample to determine if the fluorescent tag is present or not. If the signal for each individual measurement is above a threshold value, this counts as an “on” event for that measurement.

Several independent academic reviews of high-sensitivity immunoassays have highlighted that the digital approach to immunoassays allows unprecedented improvements to detection limits (see references Yeung and Cretich above).

The detection limit of the Quanterix and Merck Millipore systems is dependent upon the volume of sample that is used in the assay. For a 10 microlitre serum or plasma sample, the theoretical limit would be the detection of a single binding event, corresponding to 1 molecule. However, in terms of molarity, this would correspond to 100,000 molecules per litre of sample, or 0.16×10⁻¹⁸ moles per litre (0.16 attomolar).

However, the Quanterix and Merck Millipore systems described above are complex and cumbersome, each requiring a number of wash and transfer steps. Further, the assays can only be carried out for samples that do not contain cellular material and the systems require expensive instrumentation in order to achieve the performance that they offer. There therefore remains a need for simpler and more cost-effective high-sensitivity systems.

Additionally, the Quanterix and Merck Millipore systems become saturated at high analyte concentrations and cannot measure such high concentrations without diluting the sample.

Generally, it is challenging for an assay to have a very low detection limit and also a wide dynamic range. This is particularly the case for homogeneous assays, which do not use wash steps. Dynamic range is largely governed by the total amount of capture and reporter reagents, which is often associated with the surface area accommodating the capture and/or reporter reagent depending on the assay format. Using large surface areas with lots of reporter reagent generally leads to higher non-specific binding and background, impacting detection limit.

WO2020/260865 describes a digital assay method that uses a photosensitiser reagent to convert an optical component from a first optical state to a second optical state. However, there remains a need for optimising this system to measure both very low and very high concentrations of analyte simultaneously on the same sample, without splitting or diluting the sample.

Accordingly, the present invention provides a method for detecting an analyte in a sample suitable for detecting the analyte over a range of concentrations, the method comprising the steps of:
(i) providing the sample, a first reporter reagent and a second reporter reagent to a device, wherein each of the first and second reporter reagents comprises a photosensitiser and each of the first and second reporter reagents is capable of separately binding to the analyte, the device comprising a substrate having an optical component and a binding component, wherein the optical component and the binding component are attached to a surface of the substrate;
(ii) allowing the sample to mix with the first and second reporter reagents such that either (ii) (a) when the concentration of the analyte in the sample is less than the concentration of the first reporter reagent, a portion of the first reporter reagent binds to the analyte and the surface of the substrate in proportion to the concentration of the analyte, and a portion of the second reporter reagent binds to the surface of the substrate independently of the concentration of the analyte, or (ii) (b) when the concentration of the analyte in the sample is more than the concentration of the first reporter reagent, the first reporter reagent binds to the analyte and the surface of the substrate in proportion to the concentration of the analyte and a portion of the second reporter reagent binds to remaining analyte unbound to the first reporter reagent and the surface of the substrate in proportion to the concentration of the remaining analyte unbound to the first reporter reagent, wherein the binding to the surface of the substrate is by means of the binding component;
(iii) irradiating the device with electromagnetic radiation at one wavelength for absorption by at least the photosensitiser of the first reporter reagent, such that at least the photosensitiser of the first reporter reagent bound to the surface of the substrate absorbs electromagnetic radiation and interacts with the optical component to cause the optical component to change from a first optical state to a second optical state, thereby forming at least one set of local regions of the optical component having the second optical state on the substrate;
(iv) optionally detecting the one set of local regions of the optical component having the second optical state on the substrate formed by the first reporter reagent in step (iii) and repeating step (iii) with electromagnetic radiation at the same or a different wavelength for absorption by the photosensitiser of the second reporter reagent, such that the photosensitiser of the second reporter reagent bound to the surface of the substrate absorbs electromagnetic radiation and interacts with the optical component to cause the optical component to change from the first optical state to the second optical state, thereby forming one set of local regions of the optical component having the second optical state on the substrate; and
(v) if step (iv) is performed, detecting the one set of local regions of the optical component having the second optical state on the substrate formed by the second reporter reagent in step (iv) or, if step (iv) is not performed, detecting two sets of local regions having the second optical state on the substrate formed by the first and second reporter reagents in step (iii),
wherein the two sets of local regions are distinguishable from one another by rate of formation of the sets of local regions, order of formation of the sets of local regions, or size of local regions optionally in combination with rate of formation of the sets of local regions or order of formation of the sets of local regions.

Thus, the present invention provides a method for detecting an analyte in a sample in which only the reporter reagents in the vicinity of the surface of the substrate results in a signal, the signal being a local region of the optical component in the second optical state. It is the set of local regions of the optical component in the second optical state that are detected. Therefore, the present invention simplifies digital detection of the analyte and facilitates homogeneous assays on a range of samples, including those containing cellular materials.

Step (ii) of the method of the present invention means that at low analyte concentrations, the first reporter reagent binds preferentially to the analyte and there is non-specific binding of the second reporter reagent to the surface of the substrate, whereas at high analyte concentrations, the first reporter reagent is bound to the analyte and a portion of the second reporter reagent binds to the remaining analyte.

The fact that each of the first and second reporter reagents forms one set of local regions and the two sets of local regions are distinguishable from one another means that at low analyte concentrations, the non-specific binding of the second reporter reagent to the surface of the substrate can be distinguished from the specific binding of the first reporter reagent to the surface of the substrate and disregarded. This reduces interference and improves the accuracy and detection limit of the assay when detecting an analyte present at a low concentration in a sample.

The fact that a first reporter reagent and a second reporter reagent are used, wherein each of the first and second reporter reagents is capable of separately binding to the analyte, means that high analyte concentrations can also be detected without compromising the lower limit of detection.

Therefore, the method of the present invention increases the dynamic range of the assay, ensuring that analyte can be detected at both very low concentrations and very high concentrations. This is particularly difficult to achieve in homogenous assays that do not have multiple wash steps to help remove non-specific binding.

Therefore, the present invention provides an improved method for detecting an analyte in a sample.

The present invention will now be described with reference to the drawings, in which:

FIG. 1 shows various components which may be used in the method of the present invention;

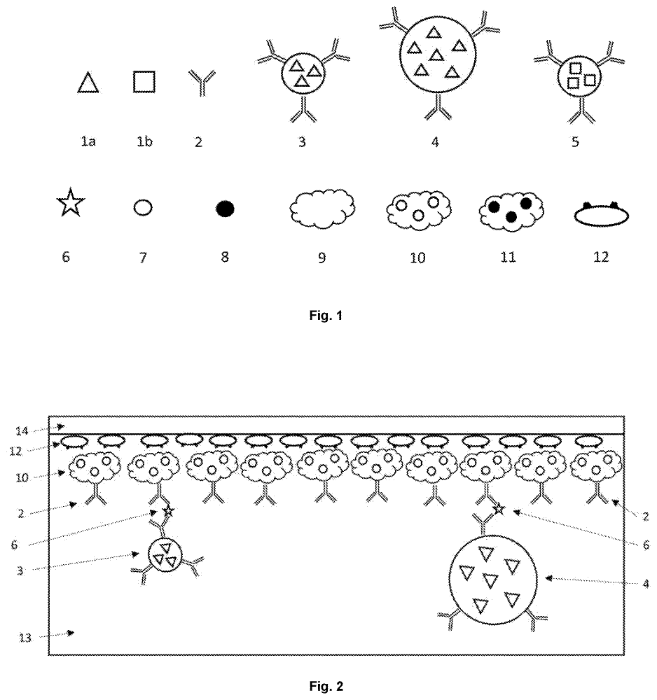

FIG. 2 shows a device in which first and second reporter reagents of different sizes are bound to the surface of the substrate before irradiation;

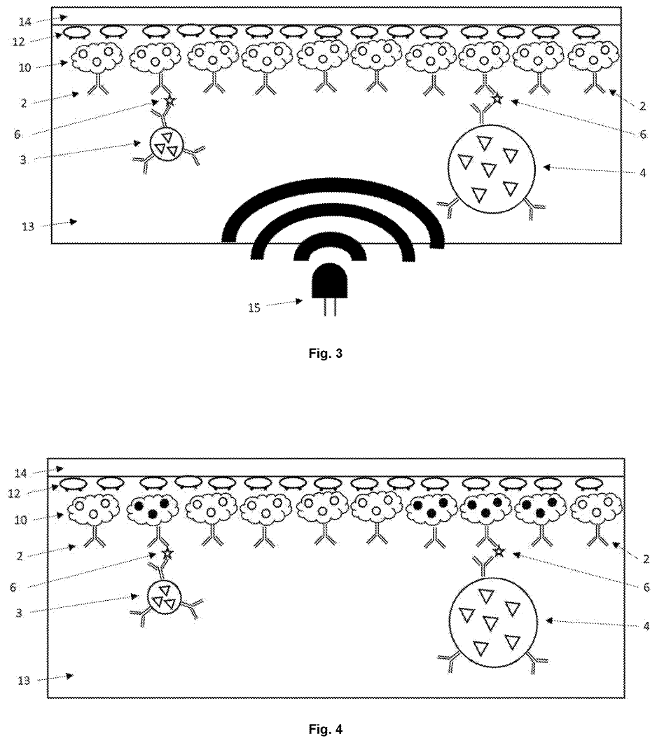

FIG. 3 shows the device of FIG. 2 being irradiated;

FIG. 4 shows the device of FIG. 3 after irradiation;

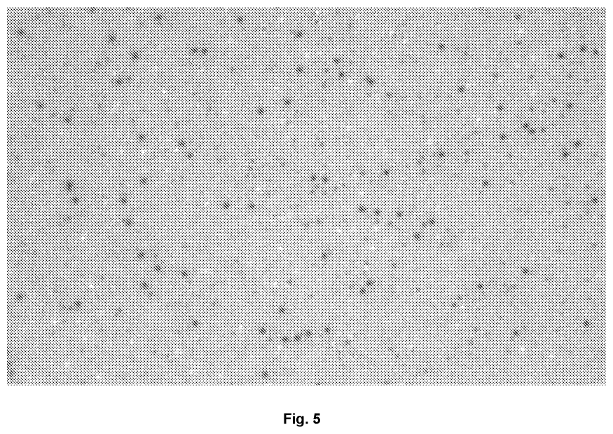

FIG. 5 shows a representative example of two sets of local regions having the second optical state on the substrate, wherein the two sets of local regions are distinguishable from one another by size of local regions, visible as differently sized dark areas bleached in a fluorescent layer;

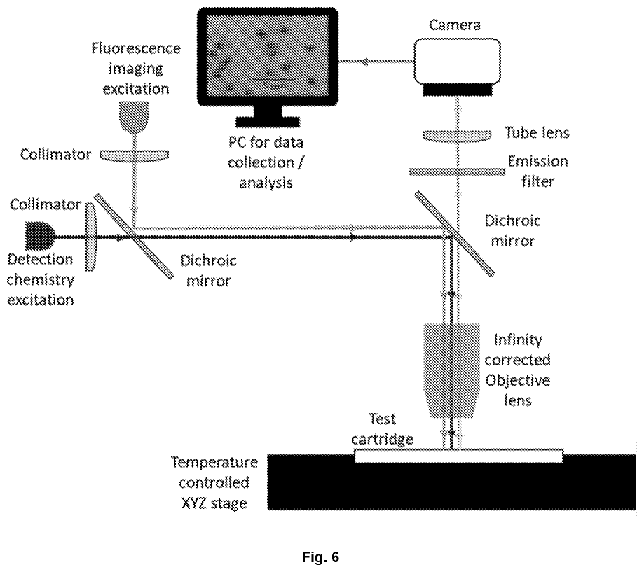

FIG. 6 shows an optical set-up for detection that allows two electromagnetic radiation sources to be focused through an objective lens;

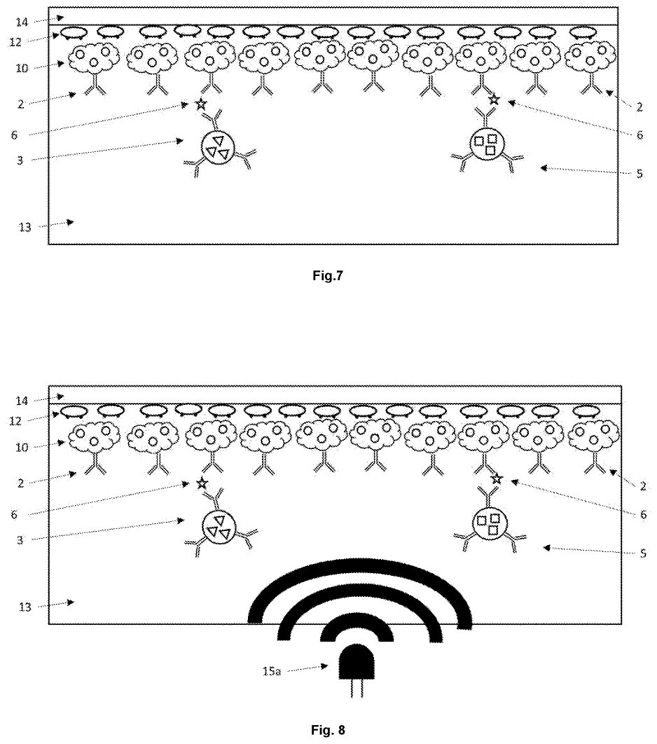

FIG. 7 shows a device in which first and second reporter reagents comprising different photosensitisers are bound to the surface of the substrate before irradiation;

FIG. 8 shows the device of FIG. 7 being irradiated at wavelength a;

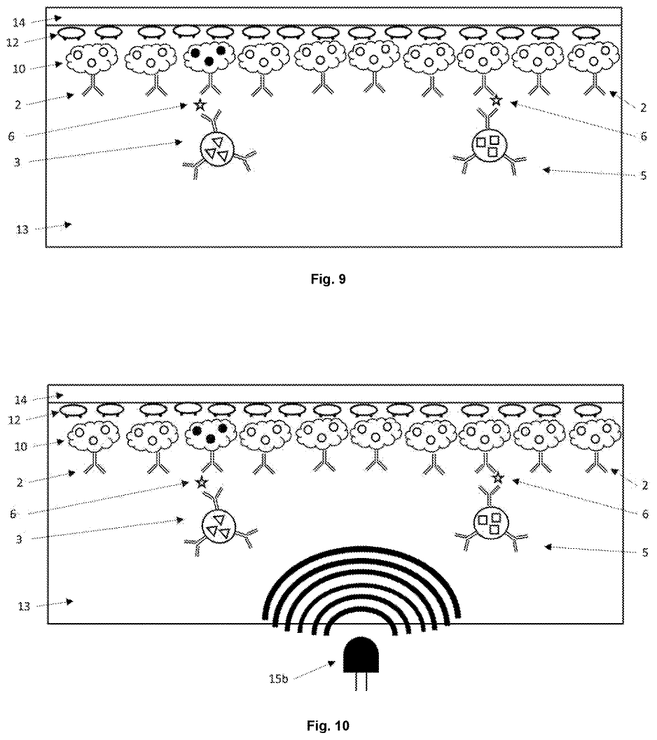

FIG. 9 shows the device of FIG. 8 after irradiation at wavelength a;

FIG. 10 shows the device of FIG. 9 being irradiated at wavelength b;

FIG. 11 shows the device of FIG. 10 after irradiation at wavelength b;

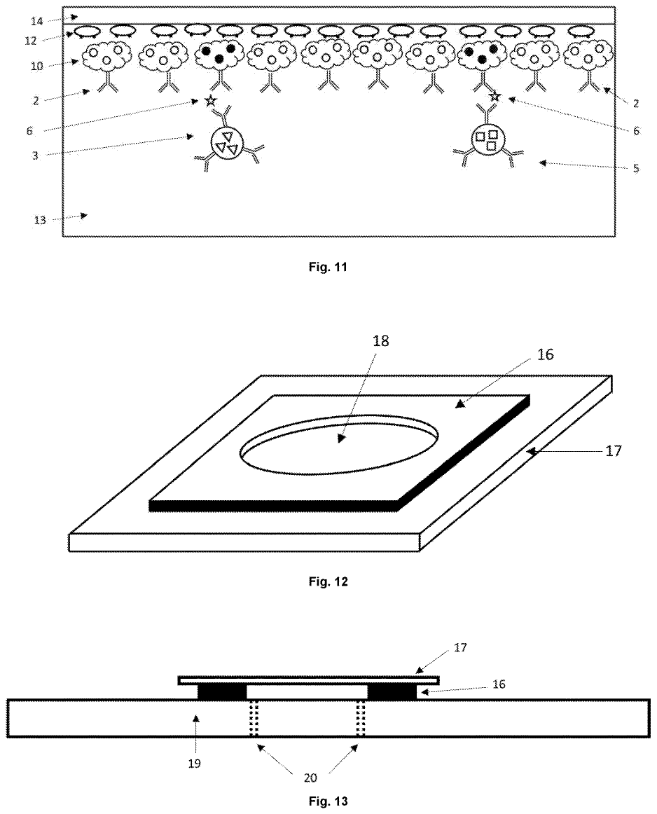

FIG. 12 shows a substrate and well created for the method of the present invention; and

FIG. 13 shows a sample chamber created using the substrate and well of FIG. 12.

The method of the present invention is used for detecting an analyte in a sample (which may be via the detection of a complex or derivative of the analyte). The method of the present invention is suitable for detecting the analyte over a range of concentrations. The concentration of the analyte can be measured over a range of six order of magnitudes or possibly even more.

The components of FIG. 1 are: photosensitisers 1a and 1b which are stimulated by different wavelengths of radiation; antibody 2; antibody-coated latex particle 3, infused with photosensitiser 1a (also referred to below as photosensitiser-labelled antibody 3); antibody-coated latex particle 4, which is different in size to particle 3 but still infused with photosensitiser 1a (also referred to below as photosensitiser-labelled antibody 4); antibody-coated latex particle 5, infused with photosensitiser 1b (also referred to below as photosensitiser-labelled antibody 5); protein analyte 6; optical component in fluorescent state 7; optical component in non-fluorescent state 8; streptavidin 9, streptavidin labelled with optical component in fluorescent state 10 (also referred to below as streptavidin-dye conjugate 10); streptavidin labelled with optical component in non-fluorescent state 11; and biotinylated BSA 12 (also referred to below as BSA-biotin conjugate 12).

Step (i) of the method of the present invention involves providing the sample, a first reporter reagent and a second reporter reagent to a device, wherein each of the first and second reporter reagents comprises a photosensitiser and each of the first and second reporter reagents is capable of separately binding to the analyte, the device comprising a substrate having an optical component and a binding component, wherein the optical component and the binding component are attached to a surface of the substrate.

The sample and the first and second reporter reagents may be mixed according to step (ii) of the method of the present invention to form a mixture comprising the sample and the first and second reporter reagents before adding the mixture to the device or the sample and the first and second reporter reagents may be added to the device sequentially and mixed according to step (ii) of the method of the present invention. The first and second reporter reagents may also be provided to the device and stored there before adding the sample and mixing according to step (ii) of the method of the present invention.

Step (ii) of the method of the present invention involves allowing the sample to mix with the first and second reporter reagents such that either (ii) (a) when the concentration of the analyte in the sample is less than the concentration of the first reporter reagent, a portion of the first reporter reagent binds to the analyte and the surface of the substrate in proportion to the concentration of the analyte, and a portion of the second reporter reagent binds to the surface of the substrate independently of the concentration of the analyte, or (ii) (b) when the concentration of the analyte in the sample is more than the concentration of the first reporter reagent, the first reporter reagent binds to the analyte and the surface of the substrate in proportion to the concentration of the analyte and a portion of the second reporter reagent binds to remaining analyte unbound to the first reporter reagent and the surface of the substrate in proportion to the concentration of the remaining analyte unbound to the first reporter reagent, wherein the binding to the surface of the substrate is by means of the binding component.

In step (ii), the sample is mixed with the first and second reporter reagents. Therefore, the sample and the first and second reporting reagents form a mixture comprising the sample and the first and second reporter reagents. The mixture typically contains additional reagents such as buffer, detergent and other additives.

The sample may be mixed with the first and second reporter reagents simultaneously or the sample may be mixed with the first and second reporter reagents sequentially.

In a preferred embodiment, the first reporter reagent is added to the sample before the second reporter reagent. This ensures that the analyte in the sample binds to the first reporter reagent before the second reporter reagent is introduced so that the dynamic range of the assay can be extended. If the first reporter reagent has a high affinity for the analyte with a slow dissociation rate (for example a half-life greater than 60 mins), the dissociation of the analyte from the first reporter reagent will be negligible in the timeframe of the assay irrespective of the affinity of the second reporter reagent for the analyte and this ensures that the first reporter reagent remains bound to the analyte.

Adding the first reporter reagent to the sample before the second reporter reagent is readily achievable in a fluidic device where analyte passes along a channel, encountering reagents sequentially, either under capillary flow or pumped conditions.

Step (ii) may be achieved by allowing the device to stand for a period of time, for example 10 minutes.

If the concentration of the analyte in the sample is less than the concentration of the first reporter reagent (i.e. at low analyte concentrations), step (ii) (a) applies and a portion of the first reporter reagent binds to the analyte and the surface of the substrate in proportion to the concentration of the analyte (specific binding of the first reporter reagent), and a portion of the second reporter reagent binds to the surface of the substrate independently of the concentration of the analyte (non-specific binding of the second reporter reagent), wherein the binding to the surface of the substrate is by means of the binding component. In this case, the sample contains bound first and second reporter reagents and unbound first and second reporter reagents free in solution. There may be a small amount of analyte free in solution because binding is an equilibrium. The portion of the second reporter reagent bound to the surface of the substrate independently of the concentration of the analyte is a small proportion of the second reporter reagent and is non-specific binding.

If the concentration of the analyte in the sample is more than the concentration of the first reporter reagent (i.e. at high analyte concentrations), step (ii) (b) applies and the first reporter reagent binds to the analyte and the surface of the substrate in proportion to the concentration of the analyte and a portion of the second reporter reagent binds to remaining analyte unbound to the first reporter reagent and the surface of the substrate in proportion to the concentration of the remaining analyte unbound to the first reporter reagent (specific binding of the first and second reporter reagents), wherein the binding to the surface of the substrate is by means of the binding component. In this case, the sample contains bound first and second reporter reagents and unbound second reporter reagent free in solution. There may be small amounts of analyte and unbound first reporter reagent free in solution because binding is an equilibrium. There may also be a small amount of second reporter reagent bound to the surface of the substrate independently of the concentration of the analyte (non-specific binding of the second reporter reagent).

The fact that each of the first and second reporter reagents forms one set of local regions and the two sets of local regions are distinguishable from one another means that at low analyte concentrations, the non-specific binding of the second reporter reagent can be distinguished from the specific binding of the first reporter reagent. This non-specific binding can be disregarded when calculating the concentration of the analyte in the sample. This reduces interference, improves the accuracy and lowers the detection limit of the assay when detecting an analyte present at a low concentration in a sample.

The fact that a first reporter reagent and a second reporter reagent are used, wherein each of the first and second reporter reagents is capable of separately binding to the analyte, means that high analyte concentrations can also be detected after the first reporter reagent has been saturated with analyte. By each of the first and second reporter reagents is capable of separately binding to the analyte is meant that once a single molecule of analyte binds to a binding domain on the reporter reagent, it does not additionally bind to another binding domain on the same or a different reporter reagent.

Therefore, the method of the present invention increases the dynamic range of the assay, ensuring that analyte can be detected at both very low concentrations and very high concentrations. This is particularly difficult to achieve in homogenous assays.

Whether step (ii) (a) or (ii) (b) applies depends on the concentration of the first reporter reagent. Preferably, the concentration of the first reporter reagent is relatively low in order to minimise non-specific binding of the first reporter reagent. The second reporter reagent will generally be at a higher concentration than the first reporter reagent in order to extend the range of the assay and prevent saturation of both reporter reagents.

By way of an explanation of the principle underlying the present invention, FIG. 2 shows a device in which first and second reporter reagents are bound to the surface of the substrate before irradiation. The device includes a substrate 14 and a sample chamber 13 for holding a sample containing protein analyte 6 dissolved or suspended therein. The substrate may be any substrate that allows detection of the two sets of local regions having the second optical state on the substrate. Preferably, the substrate is planar. Preferably, the substrate is a transparent substrate and more preferably, the substrate is glass or plastic.

The substrate 14 has antibody 2 bound to streptavidin-dye conjugate 10 attached to a surface of the substrate 14 via BSA-biotin conjugate 12. The dye acts as the optical component and the antibody acts as the binding component. BSA-biotin conjugate 12 is an inert macromolecule that facilitates attachment of the optical and binding components to the surface of the substrate 14.

Although the optical and binding components are shown in this way, any technique for holding the optical and binding components proximal to the surface of the substrate 14 is applicable. For example, the optical and binding components may be a single reagent. The optical component may also be encapsulated within a polymer layer which is coated onto the surface of the substrate 14 and the binding component attached to the polymer layer. The polymer may be silicone, polystyrene or polyisobutylene, or any other suitable polymeric plastic that can be used to encapsulate the optical component.

Alternatively, a gel layer, e.g. a hydrogel layer may be impregnated with the optical component and the gel/hydrogel layer coated onto the surface of the substrate 14 and the binding component attached to the gel/hydrogel layer.

In FIG. 2, photosensitiser-labelled antibodies 3 and 4 are bound to the surface of the substrate by means of antibody 2. Photosensitiser-labelled antibodies 3 and 4 act as the reporter reagents.

At least a portion of the first reporter reagent binds to the analyte and the surface of the substrate in proportion to the concentration of the analyte, by means of the binding component. At high analyte concentrations, the first reporter reagent binds to the analyte and the surface of the substrate in proportion to the concentration of the analyte, by means of the binding component, and a portion of the second reporter reagent binds to remaining analyte unbound to the first reporter reagent and the surface of the substrate in proportion to the concentration of the remaining analyte unbound to the first reporter reagent, by means of the binding component. For example, if the binding component and the first and second reporter reagent are antibodies, and the analyte is an antigen, the reporter reagent binds to the binding component via the analyte to form a so-called “sandwich” complex. In FIG. 2, photosensitiser-labelled antibodies 3 and 4 bind to antibody 2 via protein analyte 6. Other binding events, such as antibody-hapten binding or nucleic acid binding are also possible.

All steps up until this point have taken place in the absence of light. Step (iii) of the method of the present invention involves irradiating the device with electromagnetic radiation at one wavelength for absorption by at least the photosensitiser of the first reporter reagent, such that at least the photosensitiser of the first reporter reagent bound to the surface of the substrate absorbs electromagnetic radiation and interacts with the optical component to cause the optical component to change from a first optical state to a second optical state, thereby forming at least one set of local regions of the optical component having the second optical state on the substrate 14.

FIG. 3 shows the device of FIG. 2 being irradiated with electromagnetic radiation, preferably visible light. The light source may be, for example, LED 15. The light source illuminates the sample chamber 13 with light of the appropriate wavelength to excite the photosensitiser 1 of photosensitiser-labelled antibodies 3 and 4.

The wavelength depends on the photosensitiser. The device is typically irradiated for at least 10 seconds. Preferably, the device is irradiated with electromagnetic radiation for 1 to 30 seconds, more preferably 2 to 20 seconds, more preferably 5 to 15 seconds, and most preferably 10 seconds. This ensures that there is an irreversible optical change on the surface of the substrate, thus allowing long-lived and transient binding events to be distinguished.

FIG. 4 shows the device of FIG. 3 after irradiation. The photosensitiser 1 of photosensitiser-labelled antibodies 3 and 4 interacts with the dye optical component of the streptavidin-dye conjugate 10 to cause the dye to change from a fluorescent state to a non-fluorescent state. Streptavidin-dye conjugate in fluorescent state 10 becomes streptavidin-dye conjugate in non-fluorescent state 11. Only the dye that is in close proximity to photosensitiser 1 changes from the first optical state to the second optical state.

Alternative first and second optical states could include changes to light polarisation, fluorescence lifetime, refractive index, light scattering (including Raman scattering), phosphorescence and other optical effects.

Although FIGS. 2-4 illustrate one of each of the first and second reporter reagents bound to the surface of the substrate 14, in reality a number of the same reporter reagent will bind to the surface of the substrate 14 to create more than one local region having the second optical state on the substrate in a set of local regions having the second optical state on the substrate. Using the components of FIG. 1, these local regions having the second optical state on the substrate would be visible as discrete areas with the streptavidin-dye conjugate in non-fluorescent state 11.

Such discrete areas can be seen in FIG. 5, which shows a representative example of two sets of local regions having the second optical state on the substrate, wherein the two sets of local regions are distinguishable from one another by size of local regions, visible as differently sized dark areas bleached in a fluorescent layer. There is a clear difference in size between the sets of local regions, one set of local regions being visible as big dark areas and the other set of local regions being visible as small dark areas. The white areas are artefacts and should be ignored.

Further, the method of the present invention uses a first reporter reagent and a second reporter reagent. The first and second reporter reagents are different types of reporter reagents to form two sets of local regions having the second optical state on the substrate which are distinguishable from one another by rate of formation of the sets of local regions, order of formation of the sets of local regions, or size of local regions optionally in combination with rate of formation of the sets of local regions or order of formation of the sets of local regions.

Additionally, excess particles can be suspended in the sample that are not bound to the surface. If the sample is whole blood, then red cells will be suspended in the medium. Preferably, the substrate 14 forms the top of the sample chamber 13, allowing red blood cells to sediment away from the substrate 14.

The depth of the sample chamber 13 is designed to minimise the diffusion path length of the first and second reporter reagents, and allow the first and second reporter reagents to bind rapidly. Typically, the depth of the sample chamber is from 50 to 200 μm.

In the method of the present invention, the sample chamber 13 is filled with a sample containing an analyte. First and second reporter reagents such as photosensitiser-labelled antibodies 3 and 4 are also added to the sample chamber 13. In a preferred embodiment, the first reporter reagent is added to the sample before the second reporter reagent. If a whole blood sample is used, the sample may also contain additional components such as red blood cells.

After a period of time, at least a portion of the first reporter reagent such as photosensitiser-labelled antibody 3 is bound to the analyte and the surface of the substrate 14 in proportion to the concentration of the analyte 6 by means of a binding component such as antibody 2. At high analyte concentrations, the first reporter reagent binds to the analyte and the surface of the substrate in proportion to the concentration of the analyte, by means of the binding component, and a portion of the second reporter reagent such as photosensitiser-labelled antibody 3 binds to the remaining analyte unbound to the first reporter reagent and the surface of the substrate 14 in proportion to the concentration of the remaining analyte unbound to the first reporter reagent 6, by means of the binding component such as antibody 2.

A sufficient amount of first reporter reagent and an excess of second reporter reagent is included for the analyte of interest so that the maximum amount of analyte forms a sandwich complex. It is preferable to use a relatively small amount of the first reporter reagent in order to maximise sensitivity (by reducing non-specific binding of the first reporter reagent to the surface of the substrate) and a relatively large amount of the second reporter reagent in order to maximise the dynamic range (by preventing saturation of the second reporter reagent). In practice, the actual amounts of each reporter reagent will be assay specific and depend upon the preferred measuring range.

After irradiation, the excited photosensitiser interacts with the optical component to cause the optical component to change from a first optical state to a second optical state. For example, streptavidin labelled with optical component in fluorescent state 10 becomes streptavidin labelled with optical component in non-fluorescent state 11. Only the optical component that is in close proximity to the excited photosensitiser changes from the first optical state to the second optical state.

The photosensitiser is proximal to the substrate when the binding event has occurred. That is, the photosensitiser is sufficiently close to the surface of the substrate to interact with the optical component and convert it from the first optical state to the second optical state on irradiation of the device. The actual distance between the photosensitiser and the surface of the substrate will, however, depend on a number of variables, such as the size and nature of the photosensitiser, the size and nature of the binding components, the first and second reporter reagents and the analyte, and the nature of the sample medium.

Any excited photosensitiser in the vicinity of the optical component interacts to cause the optical component to change from the first optical state to the second optical state. In this way, at least the excited photosensitiser of the first reporter reagent bound to the surface of the substrate interacts with the optical component to cause the optical component to change from the first optical state to the second optical state, thereby forming at least one set of local regions having the second optical state on the substrate 14.

The first and second reporter reagents comprising the excited photosensitisers must be permanently bound to the surface of the substrate 14 during the entire period of irradiation in order to achieve complete conversion of the first optical state to the second optical state. If the first and second reporter reagents comprising the excited photosensitisers are only transiently bound to the surface for a fraction of the irradiation period, then there is incomplete conversion to the second optical state, which can be detected by algorithms used to measure the size, shape and intensity of the discrete areas. Any unbound first and second reporter reagents in solution does not cause the optical component to change significantly from the first optical state to the second optical state.

This provides a significant advantage over other digital assay methods in that it removes the need for washing steps. In this way, the method of the present invention is a homogeneous assay. In a conventional assay, the unbound reporter reagent must be separated from the bound reporter reagent before any measurement is taken since the unbound reporter reagent interferes with the signal generated by the bound reporter reagent. However, on account of the localised surface changes provided by the present invention, bound and unbound reporter reagents may be distinguished. Indeed, the ability to distinguish between reporter reagents proximal to the surface of the substrate 14 (i.e. bound) and reporter reagents in the bulk solution (i.e. unbound) is a particular advantage of the present invention. Preferably, steps (i) to (iii) take place in the absence of wash steps, i.e. the method is carried out without removing the sample from the substrate in steps (i), (ii) and (iii).

Step (iv) of the method of the present invention involves optionally detecting the one set of local regions of the optical component having the second optical state on the substrate formed by the first reporter reagent in step (iii) and repeating step (iii) with electromagnetic radiation at the same or a different wavelength for absorption by the photosensitiser of the second reporter reagent, such that the photosensitiser of the second reporter reagent bound to the surface of the substrate absorbs electromagnetic radiation and interacts with the optical component to cause the optical component to change from the first optical state to the second optical state, thereby forming one set of local regions of the optical component having the second optical state on the substrate.

Step (iv) is optional and occurs when the two sets of local regions having the second optical state on the substrate form sequentially in two irradiation steps and detection after each irradiation step is required.

If step (iv) is performed, detecting the one set of local regions of the optical component having the second optical state on the substrate formed in step (iii) is preferably carried out by image analysis software that can distinguish actual binding events from transient binding events and surface artefacts by analysis of surface intensity and morphology. Detection may be a separate step that is carried out after the optical component has been converted from the first optical state to the second optical state. Alternatively, detection may be carried out during the course of the conversion to the second optical state.

Any method that can successfully identify individual binding events can be used. For example, an initial image of the surface could be taken using the excitation wavelength of the optical component first, followed by irradiation with electromagnetic radiation to excite the photosensitiser, followed by a final step of irradiation with the excitation wavelength of the optical component.

Alternatively, it may be possible to capture a series of images (or a video file) of the surface whilst irradiating with electromagnetic radiation that excites both the optical component and the photosensitiser simultaneously. Any combination of optical filters, dichroic mirrors and illumination sequences that allow the binding events to be identified are acceptable.

The second optical state forms a set of local regions on the substrate. Advantageously, local regions having the second optical state can be counted as individual binding events. The method of the present invention is thus suitable for performing a digital assay. However, if there are a large number of binding events such that the majority of the optical component is in the second optical state, the bulk change may be detected.

In a preferred embodiment, the one set of local regions having the second optical state on the substrate is detected by counting local regions in the one set of local regions having the second optical state on the substrate or by measuring the one set of local regions having the second optical state on the substrate as a bulk property. More preferably, the one set of local regions having the second optical state on the substrate is detected by counting local regions in the one set of local regions having the second optical state on the substrate.

Local regions having the second optical state may need to be above or below a threshold value corresponding to a background signal, depending on the first and second optical states. For example, when the first optical state is non-fluorescent and the second optical state is fluorescent, the level of fluorescence of the second optical state may need to be above a threshold value before being detected. This would eliminate any interference from autofluorescence of the sample. Alternatively, when the first optical state is fluorescent and the second optical state is non-fluorescent, the level of fluorescence of the second optical state may need to be below a threshold value before being detected.

The local regions having the second optical state on the substrate are typically discrete areas on the substrate. However, some local regions may be excluded from detection owing to their morphology. Local regions corresponding to an individual binding event tend to be uniform and circular but some local regions may be irregular in shape, corresponding to artefacts. Further, some local regions may be larger than others where particles have clumped together, or they may be smaller, where there has only been a transient binding event. Therefore, in a preferred embodiment, only uniform, circular local regions having the second optical state on the substrate are detected.

The one set of local regions on the substrate can be detected using simple optical means. In a preferred embodiment, the one set of local regions having the second optical state on the substrate is detected using optical microscopy. An optical set-up suitable for detection is shown in FIG. 6. More preferably, the one set of local regions having the second optical state on the substrate is detected using wide-field microscopy. Wide-field microscopy is the simplest form of microscopy, whereby the whole sample is illuminated and imaged at the same time, in comparison to more complex techniques such as confocal microscopy where only one single focal spot is illuminated and recorded at a time. The benefits of confocal microscopy are increased contrast by removal of out-of-focus haze and the ability to take a stack of images through the depth of the sample. Super-resolution microscopy techniques are also known, such as photo-activated localization microscopy (PALM or FPALM) and stochastic optical reconstruction microscopy (STORM). These methods have added complexity and cost over simple wide-field methods.

In order to detect the one set of local regions on the substrate, a wide-field fluorescent microscope with a light source (for example, an LED) and a photodetector (for example a camera, such as a CCD) can be used, using excitation and emission filters suitable for the detection of the particular optical component.

If step (iv) is performed, step (iii) is repeated in the same way as irradiation step (iii), except with electromagnetic radiation at the same or a different wavelength. The preferred embodiments of step (iii) also apply to irradiating in step (iv).

In a preferred embodiment, optional step (iv) of the method of the present invention involves repeating step (iii) with electromagnetic radiation at the same wavelength. This method may be used when the first and second reporter reagents differ from one another by size, gas permeability, reactivity of the photosensitiser, amount of the photosensitiser or combinations thereof. In this case, the electromagnetic radiation used in step (iii) and optional step (iv) corresponds to the excitation wavelength of the photosensitiser of the first and second reporter reagents. In this embodiment, the two sets of local regions are distinguishable from one another by rate of formation of the sets of local regions, or size of local regions in combination with rate of formation of the sets of local regions.

Preferably, optional step (iv) of the method of the present invention involves repeating step (iii) with electromagnetic radiation at the same wavelength for a longer period of time and/or at a higher intensity. This allows the set of local regions that forms at a slower rate to then form.

In an alternative preferred embodiment, optional step (iv) of the method of the present invention involves repeating step (iii) with electromagnetic radiation at a different wavelength. This method is used when the first and second reporter reagents differ from one another by excitation wavelength of the photosensitiser optionally in combination with size. In this case, the electromagnetic radiation used in step (iii) and optional step (iv) corresponds to the excitation wavelengths of the photosensitisers of the first and second reporter reagents. In this embodiment, the two or more sets of local regions are distinguishable from one another by order of formation of the sets of local regions, or size of local regions in combination with order of formation of the sets of local regions.

Step (v) of the method of the present invention involves if step (iv) is performed, detecting the one set of local regions of the optical component having the second optical state on the substrate formed by the second reporter reagent in step (iv) or, if step (iv) is not performed, detecting two sets of local regions having the second optical state on the substrate formed by the first and second reporter reagents in step (iii), wherein the two sets of local regions are distinguishable from one another by rate of formation of the sets of local regions, order of formation of the sets of local regions, or size of local regions optionally in combination with rate of formation of the sets of local regions or order of formation of the sets of local regions.

Therefore, if step (iv) is not performed, step (iii) is irradiating the device with electromagnetic radiation at one wavelength for absorption by each of the photosensitisers of the first and second reporter reagents, such that each of the photosensitisers of the first and second reporter reagents bound to the surface of the substrate absorbs electromagnetic radiation and interacts with the optical component to cause the optical component to change from a first optical state to a second optical state, thereby forming two sets of local regions of the optical component having the second optical state on the substrate. In this instance, the sets of local regions differ by size.

Step (v) is performed in the same way as detecting in optional step (iv) except if step (iv) is not performed, two sets of local regions having the second optical state on the substrate formed by the first and second reporter reagents in step (iii) are detected rather than just one set. The preferred embodiments of detecting in optional step (iv) also apply to step (v).

In the method of the present invention, two sets of local regions having the second optical state on the substrate are detected. The two sets of local regions are distinguishable from one another by rate of formation of the sets of local regions, order of formation of the sets of local regions, or size of local regions optionally in combination with rate of formation of the sets of local regions or order of formation of the sets of local regions.

That is, the two sets of local regions are distinguishable from one another by rate of formation of the sets of local regions, order of formation of the sets of local regions or size of local regions. And if the two sets of local regions are distinguishable from one another by size of local regions, the two sets of local regions are optionally additionally distinguishable from one another by rate of formation of the sets of local regions or order of formation of the sets of local regions. That is, the two sets of local regions may be distinguishable from one another by size of local regions and rate of formation of the sets of local regions. Or the two sets of local regions may be distinguishable from one another by size of local regions and order of formation of the sets of local regions.

The difference between rate of formation and order of formation is that when the two sets of local regions are distinguishable from one another by rate of formation, the two sets of local regions will always form in the same order when using excitation at the same wavelength whereas when the two sets of local regions are distinguishable from one another by order of formation, the order of formation of the two sets of local regions can be switched by changing the order in which the two excitation wavelengths are used.

By rate of formation of the sets of local regions is meant the length of time for a set of local regions to form to a given size using electromagnetic radiation at one wavelength.

In a preferred embodiment, the two sets of local regions are distinguishable from one another by rate of formation of the sets of local regions. This is an obvious difference and at low analyte concentrations when the number of local regions in a first set of local regions is below a threshold value, the number of local regions in the first set of local regions after a first irradiation step for a short period time are counted and the number of local regions in a second set of local regions after a second irradiation step using the same wavelength for a longer period of time and/or at a higher intensity are discounted. Since the second reporter reagent does not generate any local regions during the first irradiation period, there is no interference from the second reporter reagent with the measurement using the first reporter reagent. Therefore, the non-specific binding of the second reporter reagent does not interfere with the detection of the analyte at low analyte concentrations and the accuracy and detection limit of the assay is improved.

At high concentrations of the analyte when the number of local regions in the first set of local regions is above a threshold value, both the number of local regions in the first and second sets of local regions after the first and second irradiation steps are counted. Therefore, both low and high concentrations of analyte can be detected and the dynamic range of the assay is widened.

By order of formation of the sets of local regions is meant that the sets of local regions form sequentially in response to irradiating the device with different excitation wavelengths of the photosensitisers.

In a preferred embodiment, the two sets of local regions are distinguishable from one another by order of formation of the sets of local regions. Again, this is an obvious difference and at low analyte concentrations when the number of local regions in a first set of local regions is below a threshold value, the number of local regions in the first set of local regions after a first irradiation step are counted and the number of local regions in a second set of local regions after a second irradiation step using a different wavelength are discounted. Since the second reporter reagent does not generate any local regions during the first irradiation step, there is no interference from the second reporter reagent with the measurement using the first reporter reagent. Therefore, the non-specific binding of the second reporter reagent does not interfere with the detection of the analyte at low analyte concentrations and the accuracy and detection limit of the assay is improved.

At high concentrations of the analyte when the number of local regions in the first set of local regions is above a threshold value, both the number of local regions in the first and second sets of local regions after a first irradiation step and a second irradiation steps using a different wavelength are counted. Therefore, both low and high concentrations of analyte can be detected and the dynamic range of the assay is widened.

By size of local regions is meant size of local regions at a given time or maximum size attainable, preferably maximum size attainable.

The size of each local region in a set of local regions of the optical component having the second optical state on the substrate should be the same and the local regions in one set of local regions should be clearly bigger or smaller than the local regions in the other set of local regions. Preferably, the size of each local region in a set of local regions of the optical component having the second optical state on the substrate is the same.

As discussed above, local regions corresponding to an individual binding event tend to be uniform and circular. Therefore, the two sets of local regions are preferably distinguishable from one another by diameter of local regions.

In a preferred embodiment, the two sets of local regions are distinguishable from one another by size of local regions. Again, this is an obvious difference and at low analyte concentrations when the number of local regions in a first set of local regions is below a threshold value, the number of local regions in the first set of local regions are counted and the number of local regions in a second set of local regions are discounted. Therefore, the non-specific binding of the second reporter reagent does not interfere with the detection of the analyte at low analyte concentrations and the accuracy and detection limit of the assay is improved.

At high concentrations of the analyte when the number of local regions in the first set of local regions is above a threshold value, both the number of local regions in the first and second sets of local regions are counted. Therefore, both low and high concentrations of analyte can be detected and the dynamic range of the assay is widened.

In another preferred embodiment, the two or more sets of local regions are preferably distinguishable from one another by size of local regions and rate of formation of the sets of local regions.

Alternatively, the two or more sets of local regions are preferably distinguishable from one another by size of local regions and order of formation of the sets of local regions.

The use of the first and second reporter reagents results in the two sets of local regions being distinguishable from one another by rate of formation of the sets of local regions, order of formation of the sets of local regions, or size of local regions optionally in combination with rate of formation of the sets of local regions or order of formation of the sets of local regions.

The first and second reporter reagents have different physicochemical properties. There are a number of physicochemical properties that may be varied so that the two sets of local regions are distinguishable from one another by rate of formation of the sets of local regions, order of formation of the sets of local regions, or size of local regions optionally in combination with rate of formation of the sets of local regions or order of formation of the sets of local regions. These physicochemical properties include size, material properties (e.g. gas permeability), type of photosensitiser (e.g. wavelength at which it can be activated, reactivity of photosensitiser) and/or amount of photosensitiser.

In a preferred embodiment, the first and second reporter reagents differ from one another by size, gas permeability, excitation wavelength of the photosensitiser, reactivity of the photosensitiser, amount of the photosensitiser or combinations thereof. More preferably, the first and second reporter reagents differ from one another by size, gas permeability, excitation wavelength of the photosensitiser, amount of the photosensitiser or combinations thereof. Most preferably, the first and second reporter reagents differ from one another by size and/or excitation wavelength of the photosensitiser.

Preferably, the first and second reporter reagents differ from one another by size. If the effect of the photosensitiser on the optical component is limited to a finite distance from the reporter reagent, then a larger reporter reagent can interact with the optical component at a greater distance from the centre of the reporter reagent. Additionally, a greater size allows a larger amount of photosensitiser to be associated with the reporter reagent, increasing the activity of the reporter reagent. This increased activity may facilitate a faster interaction between the photosensitiser of the reporter reagent and the optical component, leading to more rapid formation of a set of local regions, which also are greater in size. As such, the two sets of local regions are preferably distinguishable from one another by size of local regions optionally in combination with rate of formation of the sets of local regions.

In a preferred embodiment, the first and second reporter reagents differ from one another by size and the first reporter reagent is smaller than the second reporter reagent. In this case, the first reporter reagent would diffuse more rapidly to the surface of the substrate than the second reporter reagent, enabling more of the first reporter reagent to bind to the surface of the substrate in proportion to the concentration of the analyte than the second reporter reagent at low analyte concentrations for any given incubation period. The first reporter reagent would hence form more local regions of the optical component having the second optical state on the substrate than the second reporter reagent, which would be smaller for the first reporter reagent than the second reporter reagent. At high analyte concentrations, the first reporter reagent would bind to the surface of the substrate in proportion to the concentration of the analyte and a portion of the second reporter reagent would bind to the surface of the substrate in proportion to the concentration of the remaining analyte. Since the binding of the first and second reporter reagents is distinguishable by size of local regions, the dynamic range of the assay is widened. Additionally, if the second reporter reagent is larger than the first reporter reagent, the second reporter reagent can have a greater number of binding domains thus preventing saturation.

Preferably, the first and second reporter reagents differ from one another by size, affinity for the analyte and dissociation rate, wherein the first reporter reagent is smaller, has a higher affinity for the analyte and a slower dissociation rate than the second reporter reagent. When considering affinity, it is also important to consider the dissociation rate (off rate) as well as the overall affinity constants. If the first reporter reagent has a high affinity and a dissociation rate that is slow (relative to the assay duration) and the second reporter reagent has a low affinity and a dissociation rate that is rapid (relative to the assay duration) then the majority of analyte will bind to the first reporter reagent over the course of the assay duration. Further, as discussed above, the first reporter reagent would diffuse more rapidly to the surface of the substrate than the second reporter reagent. Both of these factors would enable more of the first reporter reagent to bind to the surface of the substrate in proportion to the concentration of the analyte than the second reporter reagent at low analyte concentrations. The first reporter reagent would hence form more local regions of the optical component having the second optical state on the substrate than the second reporter reagent, which would be smaller for the first reporter reagent than the second reporter reagent. At high analyte concentrations, the first reporter reagent would bind to the surface of the substrate in proportion to the concentration of the analyte and a portion of the second reporter reagent would bind to the surface of the substrate in proportion to the concentration of the remaining analyte. Since the binding of the first and second reporter reagents is distinguishable by size of local regions, the dynamic range of the assay is widened.

In a particularly preferred embodiment, the first reporter reagent is added to the sample before the second reporter reagent and the first reporter reagent is smaller than the second reporter reagent. Combining these two approaches ensures that analyte will only bind to the first reporter reagent at low analyte concentrations, thus enhancing the dynamic range of the assay.

Preferably, the first and second reporter reagents differ from one another by gas permeability. For example, greater gas permeability facilitates a faster interaction between the photosensitiser of the reporter reagent and the optical component, leading to more rapid formation of a set of local regions and/or a set of local regions having local regions of greater size. As such, the two sets of local regions are distinguishable from one another by rate of formation of the sets of local regions or size of local regions optionally in combination with rate of formation of the sets of local regions.

Preferably, the first and second reporter reagents differ from one another by excitation wavelength of the photosensitiser. As such, the two sets of local regions are distinguishable from one another by order of formation of the sets of local regions.

This can be seen in FIGS. 7-11. FIG. 7 shows a device in which first and second reporter reagents comprising photosensitisers which are stimulated by different wavelengths of radiation are bound to the surface of the substrate before irradiation. Photosensitiser-labelled antibody 3 is bound to the surface of the substrate by means of antibody 2 and photosensitiser-labelled antibody 5 is bound to the surface of the substrate by means of antibody 2. Photosensitiser-labelled antibodies 3 and 5 act as the reporter reagents.

FIG. 8 shows the device of FIG. 7 being irradiated with electromagnetic radiation at one wavelength, for example 680 nm. The light source may be, for example, LED 15a. The light source illuminates the sample chamber 13 with light of the appropriate wavelength to excite the photosensitiser 1a of photosensitiser-labelled antibody 3.

FIG. 9 shows the device of FIG. 8 after irradiation. The photosensitiser 1a of photosensitiser-labelled antibody 3 interacts with the dye optical component of the streptavidin-dye conjugate 10 to cause the dye to change from a fluorescent state to a non-fluorescent state. Streptavidin-dye conjugate in fluorescent state 10 becomes streptavidin-dye conjugate in non-fluorescent state 11. Only the dye that is in close proximity to photosensitiser 1a changes from the first optical state to the second optical state to form a set of local regions of the optical component having the second optical state on the substrate.

This set of local regions can then be detected before irradiating the device with electromagnetic radiation at a different wavelength for absorption by photosensitiser 1b of photosensitiser-labelled antibody 5.

FIG. 10 shows the device of FIG. 9 being irradiated with electromagnetic radiation at a different wavelength, for example 350 nm. The light source may be, for example, LED 15b. The light source illuminates the sample chamber 13 with light of the appropriate wavelength to excite the photosensitiser 1b of photosensitiser-labelled antibody 5.

FIG. 11 shows the device of FIG. 10 after irradiation. The photosensitiser 1b of photosensitiser-labelled antibody 5 interacts with the dye optical component of the streptavidin-dye conjugate 10 to cause the dye to change from a fluorescent state to a non-fluorescent state. Streptavidin-dye conjugate in fluorescent state 10 becomes streptavidin-dye conjugate in non-fluorescent state 11. Only the dye that is in close proximity to photosensitiser 1b changes from the first optical state to the second optical state to form an additional set of local regions of the optical component having the second optical state on the substrate.

This additional set of local regions can then be detected. As such, the two sets of local regions are distinguishable from one another by order of formation of the sets of local regions.

Preferably, the first and second reporter reagents differ from one another by reactivity of the photosensitiser. For example, the photosensitiser of one reporter reagent may absorb more electromagnetic radiation and/or interact with the optical component to a greater extent than the photosensitiser of another reporter reagent, leading to more rapid formation of a set of local regions and/or a set of local regions having local regions of greater size. As such, the two sets of local regions are distinguishable from one another by rate of formation of the sets of local regions or size of local regions optionally in combination with rate of formation of the sets of local regions.

Preferably, the first and second reporter reagents differ from one another by amount of the photosensitiser. For example, a reporter reagent containing a higher amount of photosensitiser will absorb more electromagnetic radiation and interact with the optical component to a greater extent than another reporter reagent containing less photosensitiser, leading to more rapid formation of a set of local regions and/or a set of local regions having local regions of greater size. As such, the two sets of local regions are distinguishable from one another by rate of formation of the sets of local regions or size of local regions optionally in combination with rate of formation of the sets of local regions.

The first and second reporter reagents may differ from one another by one or more than one physicochemical property.

In a preferred embodiment, the method of the present invention comprises the steps of:
(i) providing the sample, a first reporter reagent and a second reporter reagent to a device, wherein each of the first and second reporter reagents comprises a photosensitiser and each of the first and second reporter reagents is capable of separately binding to the analyte, the device comprising a substrate having an optical component and a binding component, wherein the optical component and the binding component are attached to a surface of the substrate;
(ii) allowing the sample to mix with the first and second reporter reagents such that either (ii) (a) when the concentration of the analyte in the sample is less than the concentration of the first reporter reagent, a portion of the first reporter reagent binds to the analyte and the surface of the substrate in proportion to the concentration of the analyte, and a portion of the second reporter reagent binds to the surface of the substrate independently of the concentration of the analyte, or (ii) (b) when the concentration of the analyte in the sample is more than the concentration of the first reporter reagent, the first reporter reagent binds to the analyte and the surface of the substrate in proportion to the concentration of the analyte and a portion of the second reporter reagent binds to remaining analyte unbound to the first reporter reagent and the surface of the substrate in proportion to the concentration of the remaining analyte unbound to the first reporter reagent, wherein the binding to the surface of the substrate is by means of the binding component;
(iii) irradiating the device with electromagnetic radiation at one wavelength for absorption by each of the photosensitisers of the first and second reporter reagents, such that each of the photosensitisers of the first and second reporter reagents bound to the surface of the substrate absorbs electromagnetic radiation and interacts with the optical component to cause the optical component to change from a first optical state to a second optical state, thereby forming two sets of local regions of the optical component having the second optical state on the substrate; and
(iv) detecting the two sets of local regions having the second optical state on the substrate, wherein the two sets of local regions are distinguishable from one another by size of local regions.

In this embodiment, the first and second reporter reagents preferably differ from one another by size, gas permeability, reactivity of the photosensitiser, amount of the photosensitiser or combinations thereof.

In a preferred embodiment, the method of the present invention comprises the steps of:
(i) providing the sample, a first reporter reagent and a second reporter reagent to a device, wherein each of the first and second reporter reagents comprises a photosensitiser and each of the first and second reporter reagents is capable of separately binding to the analyte, the device comprising a substrate having an optical component and a binding component, wherein the optical component and the binding component are attached to a surface of the substrate;
(ii) allowing the sample to mix with the first and second reporter reagents such that either (ii) (a) when the concentration of the analyte in the sample is less than the concentration of the first reporter reagent, a portion of the first reporter reagent binds to the analyte and the surface of the substrate in proportion to the concentration of the analyte, and a portion of the second reporter reagent binds to the surface of the substrate independently of the concentration of the analyte, or (ii) (b) when the concentration of the analyte in the sample is more than the concentration of the first reporter reagent, the first reporter reagent binds to the analyte and the surface of the substrate in proportion to the concentration of the analyte and a portion of the second reporter reagent binds to remaining analyte unbound to the first reporter reagent and the surface of the substrate in proportion to the concentration of the remaining analyte unbound to the first reporter reagent, wherein the binding to the surface of the substrate is by means of the binding component;
(iii) irradiating the device with electromagnetic radiation at one wavelength for absorption by the photosensitiser of the first reporter reagent, such that the photosensitiser of the first reporter reagent bound to the surface of the substrate absorbs electromagnetic radiation and interacts with the optical component to cause the optical component to change from a first optical state to a second optical state, thereby forming one set of local regions of the optical component having the second optical state on the substrate;
(iv) detecting the one set of local regions of the optical component having the second optical state on the substrate formed by the first reporter reagent in step (iii) and repeating step (iii) with electromagnetic radiation at the same or a different wavelength for absorption by the photosensitiser of the second reporter reagent, such that the photosensitiser of the second reporter reagent bound to the surface of the substrate absorbs electromagnetic radiation and interacts with the optical component to cause the optical component to change from the first optical state to the second optical state, thereby forming one set of local regions of the optical component having the second optical state on the substrate; and
(v) detecting the one set of local regions of the optical component having the second optical state on the substrate formed by the second reporter reagent in step (iv), wherein the two sets of local regions are distinguishable from one another by rate of formation of the sets of local regions or order of formation of the sets of local regions, and optionally size of local regions.

In this embodiment, the two or more reporter reagents preferably differ from one another by size, gas permeability, excitation wavelength of the photosensitiser, reactivity of the photosensitiser, amount of the photosensitiser or combinations thereof.

In a preferred embodiment, the method of the present invention comprises the steps of:
(i) providing the sample, a first reporter reagent and a second reporter reagent to a device, wherein each of the first and second reporter reagents comprises a photosensitiser and each of the first and second reporter reagents is capable of separately binding to the analyte, the device comprising a substrate having an optical component and a binding component, wherein the optical component and the binding component are attached to a surface of the substrate;
(ii) allowing the sample to mix with the first and second reporter reagents such that either (ii) (a) when the concentration of the analyte in the sample is less than the concentration of the first reporter reagent, a portion of the first reporter reagent binds to the analyte and the surface of the substrate in proportion to the concentration of the analyte, and a portion of the second reporter reagent binds to the surface of the substrate independently of the concentration of the analyte, or (ii) (b) when the concentration of the analyte in the sample is more than the concentration of the first reporter reagent, the first reporter reagent binds to the analyte and the surface of the substrate in proportion to the concentration of the analyte and a portion of the second reporter reagent binds to remaining analyte unbound to the first reporter reagent and the surface of the substrate in proportion to the concentration of the remaining analyte unbound to the first reporter reagent, wherein the binding to the surface of the substrate is by means of the binding component;
(iii) irradiating the device with electromagnetic radiation at one wavelength for absorption by the photosensitiser of the first reporter reagent, such that the photosensitiser of the first reporter reagent bound to the surface of the substrate absorbs electromagnetic radiation and interacts with the optical component to cause the optical component to change from a first optical state to a second optical state, thereby forming one set of local regions of the optical component having the second optical state on the substrate;
(iv) detecting the one set of local regions of the optical component having the second optical state on the substrate formed by the first reporter reagent in step (iii) and repeating step (iii) with electromagnetic radiation at the same wavelength for absorption by the photosensitiser of the second reporter reagent, such that the photosensitiser of the second reporter reagent bound to the surface of the substrate absorbs electromagnetic radiation and interacts with the optical component to cause the optical component to change from the first optical state to the second optical state, thereby forming one set of local regions of the optical component having the second optical state on the substrate; and
(v) detecting the one set of local regions of the optical component having the second optical state on the substrate formed by the second reporter reagent in step (iv), wherein the two sets of local regions are distinguishable from one another by rate of formation and optionally size of local regions.

In this embodiment, the two or more reporter reagents preferably differ from one another by size, gas permeability, reactivity of the photosensitiser, amount of the photosensitiser or combinations thereof.

In another preferred embodiment, the method of the present invention comprises the steps of:
(i) providing the sample, a first reporter reagent and a second reporter reagent to a device, wherein each of the first and second reporter reagents comprises a photosensitiser and each of the first and second reporter reagents is capable of separately binding to the analyte, the device comprising a substrate having an optical component and a binding component, wherein the optical component and the binding component are attached to a surface of the substrate;
(ii) allowing the sample to mix with the first and second reporter reagents such that either (ii) (a) when the concentration of the analyte in the sample is less than the concentration of the first reporter reagent, a portion of the first reporter reagent binds to the analyte and the surface of the substrate in proportion to the concentration of the analyte, and a portion of the second reporter reagent binds to the surface of the substrate independently of the concentration of the analyte, or (ii) (b) when the concentration of the analyte in the sample is more than the concentration of the first reporter reagent, the first reporter reagent binds to the analyte and the surface of the substrate in proportion to the concentration of the analyte and a portion of the second reporter reagent binds to remaining analyte unbound to the first reporter reagent and the surface of the substrate in proportion to the concentration of the remaining analyte unbound to the first reporter reagent, wherein the binding to the surface of the substrate is by means of the binding component;
(iii) irradiating the device with electromagnetic radiation at one wavelength for absorption by the photosensitiser of the first reporter reagent, such that the photosensitiser of the first reporter reagent bound to the surface of the substrate absorbs electromagnetic radiation and interacts with the optical component to cause the optical component to change from a first optical state to a second optical state, thereby forming one set of local regions of the optical component having the second optical state on the substrate;
(iv) detecting the one set of local regions of the optical component having the second optical state on the substrate formed by the first reporter reagent in step (iii) and repeating step (iii) with electromagnetic radiation at a different wavelength for absorption by the photosensitiser of the second reporter reagent, such that the photosensitiser of the second reporter reagent bound to the surface of the substrate absorbs electromagnetic radiation and interacts with the optical component to cause the optical component to change from the first optical state to the second optical state, thereby forming one set of local regions of the optical component having the second optical state on the substrate; and
(v) detecting the one set of local regions of the optical component having the second optical state on the substrate formed by the second reporter reagent in step (iv), wherein the two sets of local regions are distinguishable from one another by order of formation of the sets of local regions and optionally size of local regions.

In this embodiment, the two or more reporter reagents preferably differ from one another by excitation wavelength of the photosensitiser optionally in combination with size.

The photosensitiser of each of the first and second reporter reagents bound to the surface of the substrate may interact directly with the optical component to cause the change (e.g. the photosensitiser is excited and transfers this energy directly to the optical component) or indirectly with the optical component to cause the change via an additional reagent (e.g. the photosensitiser is excited and transfers this energy to an additional component, which then transfers this energy to the optical component).

In a preferred embodiment, the photosensitiser of each of the first and second reporter reagents absorbs electromagnetic radiation and interacts with a pre-activator reagent present in the mixture of the sample and the first and second reporter reagents to generate an activator reagent which interacts with the optical component to cause the optical component to change from the first optical state to the second optical state. Therefore, the first and second reporter reagents are capable of generating an activator reagent from a pre-activator reagent present in the mixture upon absorption of electromagnetic radiation, and the optical component is capable of changing from a first optical state to a second optical state on interaction with the activator reagent.

The pre-activator reagent may be present in the sample or the pre-activator reagent may be added to the mixture of the sample and the first and second reporter reagents as an additional reagent. In a preferred embodiment, the pre-activator reagent is ground state triplet oxygen. In another preferred embodiment, the activator reagent is a reactive oxygen species (ROS). Preferably, the ROS is selected from a hydroxyl radical, superoxide, peroxide, organic peroxides, peroxynitrite, singlet oxygen and mixtures thereof. More preferably, the ROS is singlet oxygen. Singlet oxygen is the preferred activator reagent because it has a short half-life and finite diffusion path length (normally less than 1 micron under aqueous conditions).

It is believed that the photosensitiser absorbs the light to generate an excited state which can undergo intersystem crossing (ISC) with oxygen present in the sample and proximal to the reporter reagent to generate singlet oxygen. Singlet oxygen then goes on to interact with the optical component as discussed below. In a particularly preferred embodiment, the pre-activator reagent is ground state triplet oxygen and the activator reagent is singlet oxygen.

Singlet oxygen has previously been used in immunoassays such as the luminescent oxygen channeling immunoassay (LOCI). The LOCI immunoassay is a homogenous non-digital assay using donor and acceptor beads that measures a bulk phenomenon. The donor beads generate singlet oxygen upon illumination at 680 nm, and the acceptor beads generate a chemiluminescent signal when activated by singlet oxygen. Binding of donor to acceptor beads is promoted by antibody-antigen binding. The reaction mixture is typically illuminated for 0.5 to 1.0 seconds and then the luminescence signal is measured for 0.5 to 1.0 seconds. Critically, the measurement takes place in the presence of all unbound donor and acceptor beads. Spatial separation of the beads minimises background signal; however, the LOCI assay cannot distinguish between long-lived and transient binding events, due to the short measurement time. The assay can achieve a detection limit of around 1-5 μg/mL for its most sensitive assays, for example interleukin 6 (IL-6) or thyroid stimulating hormone (TSH). The method of the present invention minimises background signal even further because the “donor” particles, the reporter reagent, needs to be in proximity to the surface of the substrate 11, rather than to particles in solution, in order for a signal to be detected, and must also be there for the duration of the illumination period in order to generate a signal that is above (or below) threshold criteria used in the image analysis software. Therefore, the method of the present invention is more sensitive and can detect analytes in lower concentrations than the LOCI assay.

In a preferred embodiment, the optical component is a dye. Preferably, the optical component is selected from one of the following fluorescent dyes and mixtures thereof:

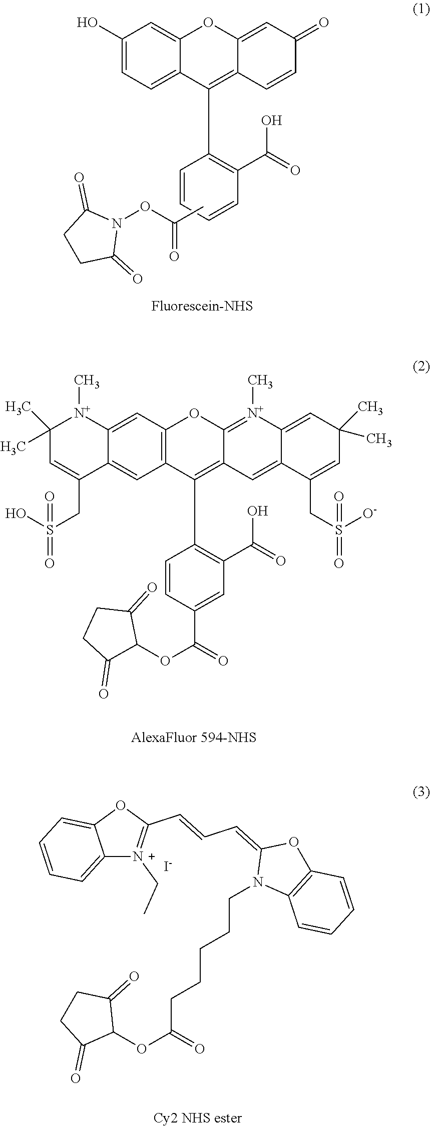

Dyes (1), (2) and (3) are common organic fluorophores that can be attached to proteins and other macromolecules by means of the attached N-hydroxy succinimide groups, which react with amine groups to form a covalent amide bond. These dyes, along with a wide range of other fluorophores can be irreversibly converted into a non-fluorescent state by the method of the present invention.

Preferably, the optical component when in one of the first and second optical states is fluorescent and the optical component when in the other of the first and second optical states is non-fluorescent. In one embodiment, the optical component in the first optical state is fluorescent and in the second optical state is non-fluorescent or the optical component in the first state is non-fluorescent and in the second optical state is fluorescent. More preferably however, the optical component when in the first optical state is fluorescent and the optical component when in the second optical state is non-fluorescent.

Preferably, the change from the first optical state to the second optical state is irreversible. This allows for subsequent scanning of the substrate to identify areas in which the change in optical state has taken place.

The assay requires the presence of a binding component.

The binding component has a binding site which is capable of binding at least a first reporter reagent proportionally to the concentration of the analyte in the sample. The proportionality is important for the functioning of the assay since the binding must be dependent on the concentration of the analyte for any meaningful measure of the concentration of the analyte to be determined. The binding may be directly proportional or indirectly proportional to the concentration of the analyte depending on the type of assay being performed. In the case of a non-competitive assay, e.g. an immunometric assay, the binding is directly proportional to the concentration of the analyte, but for a competitive assay, the binding is indirectly proportional to the concentration of the analyte.

One particular type of competitive assay is presented in which an analogue of the analyte is immobilised on the substrate, and each of the first and second reporter reagents comprises an antibody directed to the analyte. In the absence of analyte, the first and second reporter reagents would bind to the surface of the substrate. For a competitive assay, the first reporter reagent is added to the sample before the second reporter reagent or the first reporter reagent has a higher affinity for the analyte than the second reporter reagent. At low analyte concentrations, the first reporter reagent is inhibited from binding to the surface of the substrate and with increasing concentration of analyte, the binding of the first reporter reagent to the surface of the substrate decreases. At high analyte concentrations, both the first and second reporter reagents are inhibited from binding to the surface of the substrate and again, with increasing concentration of analyte, the binding of the first and second reporter reagents to the surface of the substrate decreases.

The binding component may be adapted to bind to the analyte, or a complex or derivative of the analyte, in which case the first and second reporter reagents will bind to the binding component in the presence of the analyte, or the complex or derivative of the analyte. In this case, the binding component has a binding site which is capable of binding to the first and second reporter reagents in the presence of the analyte or the complex or derivative of the analyte. The binding is, however, still proportional to the concentration of the analyte.

Alternatively, the binding component may itself be an analogue of the analyte and the first and second reporter reagents bind directly to the binding component (it is an analogue because it is bound to the surface of the substrate either through covalent bonding or non-covalent interactions). In this case, the binding component will compete with the unbound analyte, or an unbound complex or derivative of the analyte, for the binding of the first and second reporter reagents. Accordingly, the binding component will simply be capable of binding to the first and second reporter reagents.

Determining the extent of binding of the first and second reporter reagents to the binding component (either directly or mediated by the analyte/complex or derivative of the analyte) provides a measurement of the concentration of the analyte in the sample. It is customary that the system has been pre-calibrated at the point of manufacture to generate a calibration curve that is used to convert the instrument signal into the measured analyte concentration in the sample.

The assay also requires the presence of a first reporter reagent and a second reporter reagent. Each of the first and second reporter reagents comprises a photosensitiser. The photosensitiser is capable of absorbing electromagnetic radiation to interact with the optical component. It is this interaction which causes the optical component to change from the first optical state to the second optical state.

The photosensitiser may therefore be composed of any material which is capable of interacting with the electromagnetic radiation in this manner. Suitable photosensitisers are known from photodynamic therapy (PDT) as PDT reagents. PDT reagents are used in cancer therapy and dermatology to destroy cells upon illumination (Shafirstein et al., Cancers, 2017, 9, 12; Wan and Lin, Clinical, Cosmetic and Investigational Dermatology, 2014, 7, 145).

Upon irradiation with electromagnetic radiation, PDT reagents are promoted to an excited triplet state. This excited triplet state can interact directly with cellular components, in what is called a Type I process or can interact with oxygen in what is called a Type II process. Both Type I and Type II processes can lead to the formation of ROS. In the Type II process, the predominant product is singlet oxygen, through an intersystem crossing mechanism.

Singlet oxygen is an excited state of oxygen that is highly reactive. It can undergo a number of reactions before decaying, including Diels-Alder type reactions and ene reactions. It also undergoes general oxidation reactions with sulfur and nitrogen containing compounds. The indiscriminate reactivity of singlet oxygen is one of the reasons why it is used in photodynamic therapy.

A wide range of photosensitiser compounds are known, including porphyrins, chlorins (for example pyropheophorbide-a), phthalocyanines and other polyaromatic species (see, for example, Antibody-Directed Phototherapy, Pye et al., Antibodies, 2013, 2, 270).

In one embodiment, the photosensitiser is selected from porphyrins, chlorins, phthalocyanines and other polyaromatic species. In a preferred embodiment, the photosensitiser is a silicon phthalocyanine derivative, such as the example shown below:

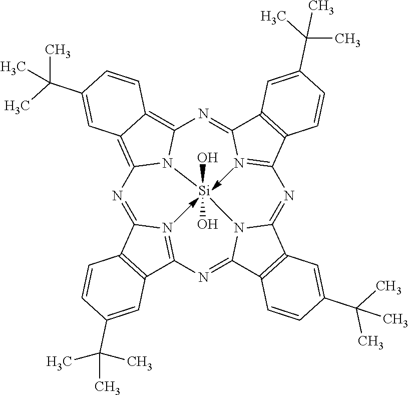

The nature of the binding component and the first and second reporter reagents will depend on the nature of the analyte, but they preferably comprise antibodies. The method of the present invention has particular applicability in immunoassays. In a particularly preferred embodiment, the binding component is an antibody raised to the analyte or the complex or derivative of the analyte, and the first and second reporter reagents comprise antibodies raised to the analyte or the complex or derivative of the analyte. In principle, a single molecule could be used for each reagent, but in practice, the binding component and the first and second reporter reagents are a population of molecules. The term “antibody” preferably includes within its scope a Fab fragment, a single-chain variable fragment (scFv), and a recombinant binding fragment.

As alternatives to antibody-antigen reactions, the binding component, the first and second reporter reagents and the analyte may be a first and second nucleic acid where the first and second nucleic acids are complementary, or reagents containing avidin or derivatives thereof and an analyte containing biotin or derivatives thereof, or vice versa. The binding component and the first and second reporter reagents may also be aptamers. The system is also not limited to biological assays and may be applied, for example, to the detection of heavy metals in water. The system also need not be limited to liquids and any fluid system may be used, e.g. the detection of enzymes, cells and viruses etc. in the air.

The photosensitiser may be on the inside or the outside of each of the first and second reporter reagents. Preferably, each of the first and second reporter reagents comprises a polymer particle and the photosensitiser is encapsulated within the polymer particle. Alternatively, each of the first and second reporter reagents further comprises a polymer particle and the photosensitiser is coated on the polymer particle. Suitable polymer particles include latex particles, generally made from polystyrene or co-polymers of polystyrene. These polymer particles can contain functional groups on the surface, such as carboxyl groups that can be used to form covalent bonds. Such polymer particles swell in non-polar solvents, allowing infusion/encapsulation of hydrophobic organic molecules.

More preferably, each of the first and second reporter reagents further comprises a polymer particle and one or more binding domains, wherein the photosensitiser is encapsulated within the polymer particle and the one or more binding domains is coated on the polymer particle. Alternatively, each of the first and second reporter reagents further comprises a polymer particle and one or more binding domains, wherein the photosensitiser and the one or more binding domains are co-coated on the polymer particle. The binding domain may be an antibody or a nucleic acid etc. as discussed above based on the nature of the analyte. Preferably however, the binding domain is an antibody.

The maximum observable signal is the maximum signal that can be achieved when monitoring the photosensitiser binding to a surface. The binding of particles to the substrate is governed by the diffusion rate of the analyte and first and second reporter reagents which is, in turn, governed largely by the hydrodynamic radius of these components and the viscosity/temperature of the sample.

The device used in the method of the present invention may further comprise controls which compensate for natural variability in the components of the measuring system, variability in the samples that are measured, and variability in the environmental conditions during the measurement. This can be achieved by exposing the sample to reagents on the surface of the substrate. The different reagents are typically located at different areas of the surface of the substrate, these areas being coated in different reagents. These controls are defined as “negative” and “positive” controls, in the sense that the negative control should approximate the expected signal in the absence of analyte, and the positive control should approximate the expected signal when analyte has saturated the system.

To achieve detection with these controls, the device of the present invention preferably comprises a binding component, a negative control reagent and a positive control reagent, each of which is attached to the surface of the substrate as described above.

The binding component is as described above.

The negative control reagent has a lower affinity for the first and second reporter reagents under the conditions of the assay than the binding component. Accordingly, the negative control reagent provides the negative control. It is important that the affinity is considered under the conditions of the assay. The reason is that in the case of a non-competitive assay, the affinity of the binding component for the reporter reagent is mediated by the presence of the analyte, or the complex or derivative of the analyte. Thus, in the absence of the analyte, or the complex or derivative of the analyte, neither the binding component nor negative control reagent has any affinity for the reporter reagent. However, in the presence of the analyte, or the complex or derivative of the analyte, the negative control reagent has a lower affinity for the reporter reagent than the binding component.

In addition, in the embodiments where the binding component binds to the analyte, or the complex or derivative of the analyte, the negative control reagent preferably has a lower affinity for the analyte or, if used, the complex or derivative of the analyte than the binding component. The negative control reagent is preferably a protein and more preferably an antibody. The negative control reagent typically has similar chemical and physical properties to the binding component, but provides little or no affinity for the reporter reagent under the conditions of the assay. In a particularly preferred embodiment, the negative control reagent has essentially no affinity for the reporter reagent under the conditions of the assay. Preferably, the negative control reagent provides essentially no affinity for the analyte or the complex or derivative of the analyte. That is, the binding of the reporter reagent, or, where applicable, the analyte or the complex or derivative of the analyte, to the negative control reagent is non-specific. In this manner, the negative control reagent can compensate for non-specific binding of the reporter reagent to the binding component. In certain embodiments, a software algorithm uses the data from the negative control region as part of the calculation to give the analyte concentration.

The positive control reagent binds to the first and second reporter reagents and has an affinity for the first and second reporter reagents which is less influenced by the concentration in the sample of the analyte or, if used, the complex or derivative of the analyte than the binding component and hence provides the positive control. Preferably, the positive control reagent has an affinity for the first and second reporter reagents which is essentially independent of the concentration of the analyte or the complex or derivative of the analyte. More preferably, the positive control reagent has a higher affinity for the first and second reporter reagents under the conditions of the assay than the binding component. In this manner, the positive control reagent measures the maximum expected signal in the system. In one embodiment, the positive control reagent may bind only to the first reporter reagent or only to the second reporter reagent. Alternatively, it may bind to both reporter reagents.

For both the negative and positive control measurements, a software algorithm may detect unusual binding patterns that could lead to an error message and abort the measurement process.

Although the description above allows the assay to incubate for a finite time before activating the photosensitiser, it is also possible that the kinetics of the binding events could be monitored by irradiating the photosensitiser for discrete periods of time over a time-course and monitoring the binding events as they take place over the course of the reaction.

The analyte may be a macromolecule or a small molecule. The macromolecule is typically a protein, such as a protein-based hormone, and may also be part of a larger particle, such as a virus, a bacterium, a cell (e.g. a red blood cell) or a prion. The small molecule may be a drug.

The term “small molecule” used herein is a term of the art and is used to distinguish the molecule from macromolecules such as proteins and nucleic acids. A small molecule is often referred to in the field of immunoassays as a “hapten”, being a small molecule which, when attached to a large carrier molecule such as a protein, can elicit an immune response and includes molecules such as hormones and synthetic drugs. A small molecule of this type will typically have a molecular weight of 2,000 or less, often 1,000 or less and even 500 or less. The binding component may be adapted to bind to the analyte itself, although the analyte can undergo a chemical reaction or initial complexing event before binding to the binding component. For example, the analyte might be protonated/deprotonated in the pH of the assay conditions. Thus, the analyte which is bound to the binding component may be the analyte itself or a derivative of the analyte; both are included within the scope of the present invention.

In a preferred embodiment, the present invention may be used to detect the presence of multiple analytes in the same sample at the same time. Different binding components could be used in different locations on the substrate for the measurement of each analyte. Sandwich and competitive assays may be run in parallel and the assays may use the same negative and positive controls as described above, or there may be separate controls for each analyte being measured.

The sample which is suspected of containing the analytes of interest will generally be a fluid sample, e.g. a liquid sample, and usually a biological sample, such as a bodily fluid, e.g. blood, plasma, saliva, serum, intraocular fluid, cerebrospinal fluid or urine. The sample may contain suspended particles and may even be whole blood. In a preferred embodiment, the sample is unprocessed and more preferably, is an unprocessed fluid. By unprocessed is meant that the sample/fluid is not pre-treated by filtration, dilution or any other pre-processing step prior to being mixed with the reporter reagent and other assay components. An advantage of the method of the present invention is that the assay may be performed on a sample which contains suspended particles without unduly influencing the results of the assay.

In a preferred embodiment, the sample is whole blood. It is surprising that the components of whole blood do not interfere with the method of detection of the present invention. It is usual to remove the red blood cells from blood to measure fluorescence in the plasma or serum component of the blood due to unpredictable scattering of light by the cellular components, which varies from sample to sample. However, in the method of the present invention, because the fluorescence would be measured on the substrate using an imaging system with a shallow depth of field, and because individual binding events can be measured, the measurement can take place in whole blood.

The sample will typically be in the order of microlitres (e.g. 1-100 μL, preferably 1-10 μL). In order to hold a fluid sample, the substrate is preferably located in a sample chamber having one or more side walls, an upper surface and a lower surface. Accordingly, the device used in the method of the present invention preferably further comprises a chamber for holding the sample containing the analyte in contact with the substrate.

A potential additional source of background interference is the settling of suspended particles on to the surface of the substrate, including first and reporter reagents and cellular components of the sample. This source of interference may be reduced by positioning the substrate above the bulk solution, e.g. on the upper surface of the reaction chamber. Thus, if any settling occurs, it will not interfere with the substrate. Preferably the substrate forms the upper surface as shown in the figures. Preferably, the substrate is substantially planar. More preferably, the substrate is planar. By “substantially planar” it is meant that the substrate deviates from planarity only up to a point where it remains functional in the invention, for example, such that the whole of the substrate remains within a single focal range when being imaged. Clearly, the optical and binding components will be on the interior surfaces of the chamber to allow contact with the sample. This and other modifications are included in the scope of the present invention.

The sample may simply be retained by surface tension forces, for example, inside a capillary channel.

The first and second reporter reagents and optionally one or more additional reagents are preferably stored in a chamber incorporated into the device used in the method of the present invention.

The method of the present invention is especially useful in point-of-care (POC) testing. POC testing is defined as diagnostic testing at or near the point of care, i.e. bedside testing. POC testing enables convenient and rapid testing, allowing improved decision making and triage whilst better allocating hospital resources such as accident and emergency care and hospital beds. This contrasts with traditional testing in which samples were taken at the point of care and then sent to the laboratory for testing. With such testing, it often takes hours or days to get the results, during which time care must continue without the desired information. POC testing often uses test kits in combination with portable instruments.

The method of the present invention is particularly useful for monitoring the concentration or presence/absence of analytes that are normally in very low abundance. Potential applications including measuring biomarkers in cardiac disease (e.g. high-sensitivity troponin), infectious disease (e.g. Hepatitis C core antigen), aging/dementia (e.g. Alzheimer's disease markers Amyloid Beta and phosphorylated Tau), cytokines and oncology (e.g. circulating tumour markers).

The present invention may also provide a device for detecting an analyte in a sample suitable for detecting the analyte over a range of concentrations, the device comprising: a substrate having an optical component and a binding component, wherein the optical component and the binding component are attached to a surface of the substrate, the optical component being capable of changing from a first optical state to a second optical state in response to interaction with irradiated photosensitisers of a first reporter reagent and a second reporter reagent bound to the surface of the substrate, wherein either (ii) (a) when the concentration of the analyte in the sample is less than the concentration of the first reporter reagent, a portion of the first reporter reagent binds to the analyte and the surface of the substrate in proportion to the concentration of the analyte, and a portion of the second reporter reagent binds to the surface of the substrate independently of the concentration of the analyte, or (ii) (b) when the concentration of the analyte in the sample is more than the concentration of the first reporter reagent, the first reporter reagent binds to the analyte and the surface of the substrate in proportion to the concentration of the analyte and a portion of the second reporter reagent binds to remaining analyte unbound to the first reporter reagent and the surface of the substrate in proportion to the concentration of the remaining analyte unbound to the first reporter reagent, wherein the binding to the surface of the substrate is by means of the binding component, thereby to form two sets of local regions having the second optical state on the substrate, wherein the two sets of local regions are distinguishable from one another by rate of formation of the sets of local regions, order of formation of the sets of local regions, or size of local regions optionally in combination with rate of formation of the sets of local regions or order of formation of the sets of local regions.

The features of the device are as described above for the device used in the method of the present invention.

In a preferred embodiment, the device further comprises a chamber for holding the sample and the first and second reporter reagents.

The device may include one or more radiation sources which are adapted to generate electromagnetic radiation corresponding to the excitation wavelengths of the photosensitisers of the first and second reporter reagents and a detector which is adapted to detect the second optical state thereby allowing a precise determination of the position of the photosensitiser with respect to the substrate.

The device may take the form of a cartridge to be used with a separate reader. The reader may incorporate the radiation source(s) and the detector. The reader is preferably a portable reader. Preferably, the device comprises a cartridge, wherein the substrate is within the cartridge, and wherein the device further comprises a detector for detecting the two sets of local regions having the second optical state on the substrate. The present invention may also provide the cartridge comprising the substrate and the optical and binding components as defined herein. The cartridge is preferably a disposable cartridge.

The present invention may also provide a system for detecting an analyte in a sample suitable for detecting the analyte over a range of concentrations, comprising: the device as described above; and the first and second reporter reagents for mixing with the sample, each of the first and second reporter reagents comprising a photosensitiser, wherein the photosensitiser is capable of absorbing electromagnetic radiation to interact with the optical component to change the optical component from the first optical state to the second optical state.

Preferably, the photosensitiser is capable of absorbing electromagnetic radiation to interact with a pre-activator reagent present in the mixture of the sample and the first and second reporter reagents to generate an activator reagent, and the activator reagent is capable of interacting with the optical component to change the optical component from the first optical state to the second optical state.

In a preferred embodiment, the system of the present invention consists essentially of the above-described features. By “essentially” is meant that no other features are required to perform the assay.

The present invention will now be described with reference to the following examples which are not intended to be limiting. These examples demonstrate binding of the first and second reporter reagents to the binding component via the analyte. However, it is to be understood that a variety of assay formats are envisaged. The examples are given to demonstrate the mode of conversion of the optical component from the first optical state to the second optical state.

### EXAMPLES

### Example 1

A reaction well as shown in FIG. 12 can be created by taking a 20×20 mm piece of polymethyl methacrylate (PMMA) of 175 micron thickness and attaching a 1 cm by 1 cm square piece of 100 μm thickness pressure sensitive adhesive (PSA) 16 with a 6 mm diameter hole cut out to the cover slip 17 to create a shallow well 18.

The surface is first coated with biotinylated BSA, then Cy2-labelled streptavidin, using methods known in the art. After coating the well with a biotinylated capture antibody (binding component), the release liner may be removed from the PSA and the substrate inverted and attached to a piece of acrylic sheet 19 with two small holes 20 drilled through to create a reaction chamber, as shown in profile in FIG. 13.

A sample is first mixed with small reporter beads (50 nm diameter) attached to a high affinity antibody for one minute, followed by addition of larger reporter beads (250 nm) coated with the same antibody. The concentration of the larger beads (particles per mL) is five times higher than the smaller beads. The two different reporter reagents are infused with the same photosensitiser. The sample is then incubated in the reaction well with a fluorescent sensor surface containing capture antibody. After eight minutes incubation, the sensor is then illuminated with red light (680 nm) for 10 seconds, then images of the fluorescent surface are captured using an objective lens/camera with excitation at 480 nm.

The fluorescent sensor surface contains discrete bleached spots where beads have bound due to the presence of analyte in the sample. The spots from the two different bead populations can clearly be distinguished by size. 50 nm beads make spots of diameter 400 nm, and the 250 nm beads makes spots that have diameter 1000 nm.

The instrument contains an algorithm that classifies and counts the spots on the surface. When the number of small spots is relatively low, the large spots are discounted, and the signal is calculated only from the small spots. Any non-specific binding of the larger beads does not interfere with the calculation of analyte concentration. When the number of small spots is above a threshold, then the larger spots are also counted and used in an algorithm that calculates the analyte concentration.

### Example 2

The assay is run in the same way as Example 1, except that the first and second reporter reagents are the same size which are activated using different excitation wavelengths. The first reporter reagent is excited at wavelength 680 nm and the second reporter reagent is excited at wavelength 350 nm.

The sample is mixed with the first reporter reagent first, as above. The second reporter reagent is in five-fold excess. After incubation, the sensor is illuminated at wavelength 680 nm and spots are counted, then wavelength 350 nm, where additional spots are counted.

1. A method for detecting an analyte in a sample suitable for detecting the analyte over a range of concentrations, the method comprising the steps of:
(i) providing the sample, a first reporter reagent and a second reporter reagent to a device, wherein each of the first and second reporter reagents comprises a photosensitiser and each of the first and second reporter reagents is capable of separately binding to the analyte, the device comprising a substrate having an optical component and a binding component, wherein the optical component and the binding component are attached to a surface of the substrate;
(ii) allowing the sample to mix with the first and second reporter reagents such that either (ii) (a) when the concentration of the analyte in the sample is less than the concentration of the first reporter reagent, a portion of the first reporter reagent binds to the analyte and the surface of the substrate in proportion to the concentration of the analyte, and a portion of the second reporter reagent binds to the surface of the substrate independently of the concentration of the analyte, or (ii) (b) when the concentration of the analyte in the sample is more than the concentration of the first reporter reagent, the first reporter reagent binds to the analyte and the surface of the substrate in proportion to the concentration of the analyte and a portion of the second reporter reagent binds to remaining analyte unbound to the first reporter reagent and the surface of the substrate in proportion to the concentration of the remaining analyte unbound to the first reporter reagent, wherein the binding to the surface of the substrate is by means of the binding component;
(iii) irradiating the device with electromagnetic radiation at one wavelength for absorption by at least the photosensitiser of the first reporter reagent, such that at least the photosensitiser of the first reporter reagent bound to the surface of the substrate absorbs electromagnetic radiation and interacts with the optical component to cause the optical component to change from a first optical state to a second optical state, thereby forming at least one set of local regions of the optical component having the second optical state on the substrate;
(iv) optionally detecting the one set of local regions of the optical component having the second optical state on the substrate formed by the first reporter reagent in step (iii) and repeating step (iii) with electromagnetic radiation at the same or a different wavelength for absorption by the photosensitiser of the second reporter reagent, such that the photosensitiser of the second reporter reagent bound to the surface of the substrate absorbs electromagnetic radiation and interacts with the optical component to cause the optical component to change from the first optical state to the second optical state, thereby forming one set of local regions of the optical component having the second optical state on the substrate; and
(v) if step (iv) is performed, detecting the one set of local regions of the optical component having the second optical state on the substrate formed by the second reporter reagent in step (iv) or, if step (iv) is not performed, detecting two sets of local regions having the second optical state on the substrate formed by the first and second reporter reagents in step (iii),
wherein the two sets of local regions are distinguishable from one another by rate of formation of the sets of local regions, order of formation of the sets of local regions, or size of local regions optionally in combination with rate of formation of the sets of local regions or order of formation of the sets of local regions.

2. The method as claimed in claim 1, wherein the first reporter reagent is added to the sample before the second reporter reagent.

3. The method as claimed in claim 1, wherein the first and second reporter reagents differ from one another by size, gas permeability, excitation wavelength of the photosensitiser, reactivity of the photosensitiser, amount of the photosensitiser or combinations thereof.

4. The method as claimed in claim 3, wherein the first and second reporter reagents differ from one another by size and the first reporter reagent is smaller than the second reporter reagent.

5. The method as claimed in claim 1, wherein step (iii) is
irradiating the device with electromagnetic radiation at one wavelength for absorption by each of the photosensitisers of the first and second reporter reagents, such that each of the photosensitisers of the first and second reporter reagents bound to the surface of the substrate absorbs electromagnetic radiation and interacts with the optical component to cause the optical component to change from a first optical state to a second optical state, thereby forming two sets of local regions of the optical component having the second optical state on the substrate;
step (iv) is not performed; and
in step (v), the two sets of local regions are distinguishable from one another by size of local regions.

6. The method as claimed in claim 1, wherein step (iii) is
irradiating the device with electromagnetic radiation at one wavelength for absorption by the photosensitiser of the first reporter reagent, such that the photosensitiser of the first reporter reagent bound to the surface of the substrate absorbs electromagnetic radiation and interacts with the optical component to cause the optical component to change from a first optical state to a second optical state, thereby forming one set of local regions of the optical component having the second optical state on the substrate;
step (iv) is performed;
in step (v), the two sets of local regions are distinguishable from one another by rate of formation of the sets of local regions, order of formation of the sets of local regions, or size of local regions in combination with rate of formation of the sets of local regions or order of formation of the sets of local regions.

7. The method as claimed in claim 1, wherein each of the first and second reporter reagents further comprises a polymer particle and the photosensitiser is encapsulated within the polymer particle.

8. The method as claimed in claim 1, wherein the photosensitiser of each of the first and second reporter reagents absorbs electromagnetic radiation and interacts with a pre-activator reagent present in the mixture of the sample and the first and second reporter reagents to generate an activator reagent which interacts with the optical component to cause the optical component to change from the first optical state to the second optical state.

9. The method as claimed in claim 8 wherein the activator reagent is a reactive oxygen species.

10. The method as claimed in claim 9, wherein the reactive oxygen species is singlet oxygen.

11. The method as claimed in claim 1, wherein the first optical state is fluorescent and the second optical state is non-fluorescent.

12. The method as claimed in claim 1, wherein the change from the first optical state to the second optical state is irreversible.

13. The method as claimed in claim 1, wherein steps (i) to (iii) take place in the absence of wash steps.

14. The method as claimed in claim 1, wherein the sample is unprocessed.

15. The method as claimed in claim 1, wherein the device is irradiated with electromagnetic radiation for more than 1 second.
